(19) United States
(12) Patent Application Publication
(10) Pub. No.: US 2026/0240967 A1
(43) Pub. Date: Aug. 20, 2026

(54) TREATMENT OF ATOPIC DERMATITIS USING SELF-REPLICATING RNA EXPRESSING IL-31

(71) Applicant: CEVA SANTE ANIMALE, Libourne (FR)

(72) Inventors: Cecile CHALLIER, Libourne (FR); Kurt KAMRUD, Lenexa, KS (US); Jason ROTH, Lenexa, KS (US); Claudine ZEMIRLINE, Libourne (FR)

(73) Assignee: CEVA SANTE ANIMALE, Libourne (FR)

(21) Appl. No.: 19/111,198

(22) PCT Filed: Sep. 14, 2023
(86) PCT No.: PCT/IB2023/000551

§ 371 Date: Mar. 12, 2025

Related U.S. Application Data
(60) Provisional application No. 63/375,602, filed on Sep. 14, 2022.

(51) Int. Cl. A61K 39/00; A61K 39/385; A61P 17/04; C07K 14/005; C07K 14/54; C07K 14/715; C12N 9/10; C12N 15/85; C12N 15/88

(52) U.S. Cl. CPC A61K 39/001; A61K 39/385; A61P 17/04; C07K 14/005; C07K 14/54; C07K 14/7155; C12N 9/1085; C12N 15/85; C12N 15/88; A61K 2039/53; A61K 2039/552; A61K 2039/6037; C07K 2319/55; C12N 2770/36134; C12N 2830/50; C12N 2840/203; C12Y 205/01078

(57) ABSTRACT

A self-replicating saRNA expressing IL-31 or its fragments or variants, IL31RA, Oncostatin M receptor β, compositions formulated for administering the saRNA to a subject in need thereof, and methods for treating atopic dermatitis and other IL-31 related conditions with the saRNA.

### CONTINUITY DATA

This application is the U.S. national stage of International Application No. PCT/IB2023/000551, filed Sep. 14, 2023, which claims priority to U.S. Provisional Application No. 63/375,602, filed Sep. 14, 2022, and which is incorporated by reference for all purposes.

### REFERENCE TO A SEQUENCE LISTING

In accordance with 37 CFR § 1.833-1835 and 37 CFR§ 1.77(b)(5), the specification makes reference to a Sequence Listing submitted electronically as a .xml file named “REPLACEMENT_012726_557313US”. The .xml file was generated on Jan. 27, 2025, and is 20,969 bytes in size. The entire contents of the Sequence Listing are hereby incorporated by reference.

### BACKGROUND OF THE INVENTION

Field of the Invention. The invention pertains to the fields of veterinary medicine and immunotherapy of diseases, disorders, or conditions associated with or mediated by Interleukin-31 (IL-31), including pruritus, alopecia, and dermatitis. It relates to a vaccine or biologic that attenuates IL-31 signaling, for example, by inducing immune responses which neutralize or otherwise attenuate the effects of IL-31 or block its interaction with its receptor thus ameliorating diseases, disorders, or conditions induced by or aggravated by IL-31.

Description of Related Art. Interleukin-31 (IL-31) is a member of the gp130/interleukin-6 cytokine family that is produced by cell types such as T helper 2 (TH2) lymphocytes and cutaneous lymphocyte antigen positive skin homing T cells. It is a potent pruritogenic cytokine and its systemic and local administration induces scratching behavior in dogs, rodents, horses, and monkeys.

Signaling. IL-31 and its receptors are involved in eczema and atopic dermatitis, especially during the acute phase. IL-31 is preferentially produced by Th2 cells and is overproduced in atopic dermatitis. See Andrea J Gonzales, et al., Interleukin-31: its role in canine pruritus and naturally occurring canine atopic dermatitis. VET. DERMATOL 2013, 24: 48-12; and Mohammed D. Saleem et al. Interleukin-31 pathway and its role in atopic dermatitis: a systematic review, JOURNAL OF DERMATOLOGICAL TREATMENT, 2017, Volume 28, Issue 7.

IL-31 binds to a receptor complex comprising IL31RA (a gp130-like receptor) IL31RA that heterodimerizes with the oncostatin M receptor β (OSMRβ). Downstream intracellular signaling involves the activation of Janus kinase-signal transducer and activator of transcription (JAK-STAT), mitogen-activated protein kinase (MAPK), and phosphatidylinositol 3-kinase (PI3K) pathways.

Over-expression. Over-expression of IL-31 induces clinical and histological features identical to those of atopic dermatitis, notably pruritus. Injection of canine Interleukin 31 (cIL-31) into laboratory beagle dogs caused transient episodes of pruritic behavior and cIL-31 has been detected in dogs with naturally occurring atopic dermatitis (AD). Expression of equine IL-31 has been associated with skin lesions and insect bite hypersensitivity in horses. In mice, upregulated expression of IL-31 is associated with atopic dermatitis. In humans, IL-31 serum levels correlate with the severity of atopic dermatitis in adults and children.

Description of atopic dermatitis and other conditions associated with dysregulated expression of IL-31. The cytokine interleukin-31 has been implicated in the pathophysiology of multiple atopic disorders such as atopic dermatitis (AD), allergic rhinitis, and airway hyper-reactivity.

Atopic dermatitis (AD), also known as atopic eczema, is a long-term type of inflammation of the skin (dermatitis). Atopic dermatitis (AD) is a chronic or chronically relapsing, eczematous, severely pruritic skin disorder associated with skin barrier dysfunction. Atopic dermatitis results in itchy, red, swollen, and cracked skin or hair loss. Clear fluid may come from the affected areas, which often thickens over time. During atopic dermatitis an itch-scratch cycle may ensue where a strong action of scratching facilitates susceptibility to a vicious cycle of itching and exacerbation of skin lesions. Loss of weight may also accompany atopic dermatitis. Treatment may result in amelioration of these symptoms.

IL-31 is one of the main drivers of a cardinal symptom of atopic dermatitis: pruritus. The lesioned skin of AD exhibits T helper 2 (T_{H} 2)-deviated immune reactions as noted above IL-31 is preferentially produced by T_{H} 2 cells.

Canines. Canine Atopic Dermatitis (cAD) is described as a relapsing pruritic inflammatory skin disease characterized by a major immune dysregulation, notably by an overproduction of Th2 cytokines. This major cytokine dysregulation is characterized mainly by an overproduction of proinflammatory cytokines including IL-31, a pruritogen cytokine; IL-4 and IL-13, cytokines which are mainly involved in inflammation. Other cytokines such as IL-33, IL-22, and TSLP may also affect or aggravate canine atopic dermatitis. In some embodiments, antibodies to these other cytokines may be administered in combination with the IL-31 or IL31 receptor vaccines disclosed herein.

Marines. When overexpressed in transgenic mice, IL-31 induces severe pruritus, alopecia and skin lesions. Moreover, IL-31 as well as its receptors are up-regulated in murine models of atopic dermatitis where animals display scratching behaviors. Transgenic mice overexpressing IL-31 develop strong pruritus, as well as skin lesions often with hair loss, which are histologically characterized by increased inflammatory cell infiltration. Intradermal injection of IL-31 was further shown to induce itching and consecutive scratching in murine wild-type or previously unaffected skin.

Equines. Insect bite hypersensitivity (IBH) is the most common seasonal pruritic allergic dermatitis of horses occurring upon insect bites. IL-31 was detectable in skin lesions of insect-bite affected horses; Olomski, F., et al, Interleukin 31 in insect bite hypersensitivity-Alleviating clinical symptoms by active vaccination against itch ALLERGY, 2020, 75(4):862-871.

Existing treatments for atopic dermatitis. Various strategies have been proposed to treat conditions mediated by IL-31. Existing treatments rely on two strategies: administration of anti-IL-31 monoclonal antibodies and use of JAK inhibitors. There are no commercialized RNA-based vaccines against IL-31.

IL-31 antagonists. IL-31 is the target of several biologics or drugs on the market for treatment of atopic dermatitis. These include with therapeutics based on anti-IL-31 monoclonal antibodies for passive immunization and JAK signal transduction inhibitors. Monoclonal antibodies that bind to IL-31 are described by U.S. Pat. No. 8,790,651 and by EP3219729 and are found in CYTOPOINT® (lokivetmab) which is a canine IL-31 monoclonal antibody licensed for treating clinical symptoms of atopic dermatitis in dogs. JAK inhibitors, which interfere with IL-31 signal transduction, are found in APOQUEL® (oclacitinib) which is licensed for treating clinical symptoms of atopic dermatitis in dogs. However, the drawback of the existing passive-immunization and drug therapies is that frequent administration of an anti-IL-31 antibody or drug, like a JAK inhibitor, is required. For example, CYTOPOINT® (lokivetmab) should be administrated once a month and APOQUEL® should be administrated twice daily. In addition, another drawback with APOQUEL® is that this drug has side effects that weaken the immune system; it may increase susceptibility to infection and exacerbate neoplastic conditions. Dogs receiving APOQUEL® tablets should therefore be monitored for the development of infections and neoplasia.

There are significant limitations and disadvantages to using treatments such as CYTOPOINT® and APOQUEL®. After administration of CYTOPOINT® the levels of the administered monoclonal antibodies decrease quickly necessitating multiple repeat injections every month. Thus, CYTOPOINT® does not provide a long lasting efficacy against atopic dermatitis.

APOQUEL® is administered once or twice daily. It modulates the subject's immune system and can increase the risk of infection or exacerbate neoplasms. According to its safety information, APOQUEL® should not be used in dogs less than 12 months of age or those with serious infections. APOQUEL® may increase the chances of developing serious infections and may cause existing parasitic skin infestations or pre-existing cancers to get worse. New neoplastic conditions (benign and malignant) were observed in clinical studies.

In contrast to CYTOPOINT® and APOQUEL® an IL-31 vaccine as disclosed by the inventors requires fewer injections, for example a priming dose, a booster dose, and then an annual booster dose, and is more economical and convenient to use.

Presently there is no licensed drug or biologic for treatment of atopic dermatitis based on active immunization with IL-31 or with its peptide epitopes via self-replicating RNA or based on active immunization or administration of IL-31 receptor (IL31RA) and Oncostatin M receptor beta) or its components. There is a need for alternative therapies based on an active immunization with a vaccine against IL-31 or its receptor, which can induce a sustained neutralizing antibody response against these targets with reduced number of injections compared to existing therapies.

With an objective to overcome the drawbacks of existing therapeutics, the inventors developed a self-replicating alphavirus saRNA that encodes IL-31 or its epitopes or that encodes an IL-31 receptor or its components or epitopes. This saRNA when administered to a subject increases the expression of IL-31, IL-31 fragments, or IL-31 variants (including variants with no or reduced IL-31 activity) that can induce an immune response against IL-31 or its receptor, thereby reducing levels of IL-31 or activation via IL-31 in a subject having or at risk of having dermatitis.

### BRIEF SUMMARY OF THE INVENTION

One aspect of this technology is a self-replicating RNA or saRNA that encodes and expresses IL-31, its fragments, and variants. Advantageously, the saRNA is based on an alphavirus platform that expresses alphavirus non-structural proteins, such as nsp1-nsp4, into which RNA encoding IL-31, its fragments, or variants, is incorporated, especially RNA encoding humoral or cellular IL-31 or IL-31 receptor epitopes. In some embodiments, the saRNA comprises additional elements which further enhance the expression of IL-31, its fragments, or variants. In some embodiments, the saRNA expresses additional protein components, such as immunogenic carrier proteins or immunomodulators which enhance recognition of IL-31 epitopes expressed by the saRNA. It may also comprise protein components, such as signal sequences or leader peptides that traffic expressed IL-31 components to different parts of a cell; see <hypertext transfer protocol secure://alg.manifoldapp.org/read/fundamentals-of-cell-biology/section/92527ecb-306c-4002-a411-e3c5d5da5e6a> (last accessed Jul. 15, 2022, incorporated by reference).

Another aspect of this technology is a method for preventing or treating eczema, atopic dermatitis, or other conditions caused or aggravated by IL-31, by administering the self-replicating RNA or saRNA to a subject in need thereof and thereby reducing the level of IL-31 or IL-31 receptor activation, increasing the clearance or inactivation of IL-31, decreasing the activity of IL-31 in the subject, or decreasing the ability of IL-31 to functionally bind to its receptors, thereby preventing or ameliorating eczema, atopic dermatitis or conditions associated with IL-31.

A related aspect of this technology is a composition comprising the self-replicating RNA or saRNA and a pharmaceutically acceptable carrier and optionally other medications, such as anti-inflammatory drugs or antihistamines, biologics such as monoclonal antibodies to other cytokines, suitable for reducing the severity of eczema, atopic dermatitis or other IL-31 associated diseases, disorders or conditions.

Other aspects of this technology involve methods for constructing a self-replicating saRNA or DNA vectors expressing the saRNA, as disclosed herein.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings below.

FIG. 1 depicts an saRNA based on an alphavirus scaffold comprising IL-31 coding sequences for IL-31, its epitopes, its fragments, or variants.

5′ CAP 5′ UTR nsPs 1-4 26s promoter IL-31 coding sequences 3′ UTR
polyA tail opt. terminator

FIG. 2 depicts an saRNA based on an alphavirus scaffold comprising IL-31 coding sequences for IL-31, its epitopes or variants, with reduced or no IL-31 activity. The IL-31 sequences with reduced activity typically comprise one or more epitopes of IL-31 capable of inducing a host immune response against IL-31 which reduces the severity of atopic dermatitis. Modification of the IL-31 coding sequences include truncation, addition of residues, or substitution of amino acid residues, especially those in or near the active site residues, with other amino acid residues.

5′ CAP 5′ UTR nsPs 1-4 26s promoter IL-31 coding sequences 3′
UTR polyA tail opt. terminator
(IL-31 reduced activity)

FIG. 3 depicts a saRNA or variants of IL-31 which are flanked on one or both ends by sequences encoding immunogenic or immunomodulatory protein segments (***). The residues of the immunogenic protein or carrier protein encoded and expressed by the saRNA may be fused or unfused to the IL-31 sequences.

5′ CAP 5′ UTR nsP1-nsP4 26s promoter *** IL-31 coding sequences ***
3′ UTR poly-A opt. terminator
(*** coding sequence for immunogenic protein)

FIG. 4 depicts an saRNA based on an alphavirus scaffold comprising IL-31 sequences encoding one or more epitopes or variants of IL-31. Sequences encoding trafficking residues (###) are incorporated near the beginning and/or end of the IL-31 coding sequences. These help traffic saRNA-expressed IL-31 polypeptides to the cytoplasm, to the endoplasmic reticulum and Golgi, into the supernatant, or into a cell membrane. IL-31 has a signal sequence that naturally traffics it to the ER and Golgi. In some embodiments, IL-31, its epitopes, fragments or variants are expressed without a signal sequence, or with other trafficking sequences.

5′ CAP 5′ UTR nsP1-nsP4 26s promoter ### IL-31 coding sequence ###
3′ UTR poly-AS tail opt. terminator
(### coding sequence for trafficking residues)

FIG. 5A depicts an saRNA based on an alphavirus scaffold comprising IL-31 sequences encoding IL-31, one or more epitopes of IL-31, IL-31 fragments, or IL-31 variants; and a DLP loop for enhanced expression of IL-31 polypeptides.

5′ CAP 5′ UTR nsP1-nsp4 26s promoter DLP loop IL-31 coding sequence
3′ UTR poly-A tail opt. terminator

FIG. 5B depicts an saRNA based on an alphavirus scaffold comprising IL-31 sequences encoding one or more epitopes of IL-31 and a DLP loop for enhanced expression of IL-31 polypeptides.

5′CAP 5′UTR DLP loop nsP1-nsP4 26s promoter IL-31 coding sequence
3′UTR poly-A tail opt. terminator

FIG. 6 depicts an saRNA based on an alphavirus scaffold comprising sequences encoding IL-31, one or more epitopes of IL-31, IL-31 fragments, IL-31 variants; and an IRES for enhanced expression of encoded IL-31 or other encoded proteins. IREs may also be placed upstream of other non-IL-31 coding sequences such as those for immunogenic or immunomodulatory proteins or for trafficking residues.

5′ CAP 5′ UTR nsP1-nsp4 26s promoter IRES IL-31 coding sequence
3′ UTR poly-A tail opt. terminator

FIG. 7A depicts a three dimensional model of the IL-31 cytokine 4-alpha helix bundle denoting sites 2 and 3. Expression of IL-31 is associated with pruritis.

FIG. 7B depicts Oncostatin M receptor β (OSMR) on the left and IL31RA on right and indicates interactions of IL-31 with these receptors.

FIG. 8 illustrates some murine IL-31 constructs: mIL31 (murine IL31), mIL31m (mIL31 with K138A substitutional mutation); mIL31-LS-V2 (murine IL31 fused to lumazine synthase) with His-Strep tags at C terminus; mIL31m-LS-V2 (inactivated murine IL31 due to K138A mutation fused to lumazine synthase) with His-Strep tags at C terminus; mDip-mIL31 (mutated diphtheria toxin or toxoid fused to murine IL31; and mDip-mIL31 (mutated diphtheria toxin or toxoid fused to murine IL31 with K138A mutation.

FIGS. 9A, 9B, 10A and 10B show that there was little or no detection of IL-31 expression in cell supernatant with the constructs encoding a portion of diphtheria toxoid. Expression was only detected in the cell membrane fraction.

FIG. 9A shows that IL-31 was expressed and fluorescently detected in the first four constructs in the supernatant, but no expression was detected in the supernatant for the two constructs (mDip-mIL31, mDip-mIL31(m)) encoding mutated diphtheria toxin or by the cell control.

FIG. 9B shows a western blot of proteins expressed by the constructs shown in FIG. 8.

FIG. 10A shows fluorescent detection of mIL31 or mIL31(m) in cells containing the mDip-mIL31 or mDip-mIL31(m) constructs. Low levels of IL-31 expression were seen.

FIG. 10B shows a western blot of supernatant, cytoplasm, or membrane fractions of cells containing the mDip-mIL31 or mDip-mIL31(m) constructs.

FIG. 11 depicts some canine IL-31 constructs, such as those described in Example 4. The final two GOIs include fusing the 3′ end of the inactivated Diphtheria toxin ORF to the 5′ end of each IL-31 ORF (native and inactivated independently) with the IL-31 signal peptide removed

FIG. 12 presents the results from Example 3. As shown, significant differences were seen between mice vaccinated with the described constructs compared to the negative and positive controls not receiving these constructs. PV: no vaccine only PBS, no IL-31 challenge; P+IL-31: no vaccine only PBS with subsequent IL-31 challenge. Vacc1+IL31: construct encodes wild-type murine IL-31 with subsequent IL-31 challenge; Vacc2+IL31: construct encodes wild-type murine IL-31 fused to Lumazine synthase with subsequent IL-31 challenge; Vacc3+IL31: construct encodes wild-type murine IL-31 fused to Diphtheria toxoid with subsequent IL-31 challenge. As apparent, Vaccines 2 and 3 provided superior results compared to Vaccine 1 that was not fused to either Lumazine synthase or to diphtheria toxoid.

### DETAILED DESCRIPTION OF THE INVENTION

### Definitions

Eczema (also atopic eczema or atopic dermatitis) is a general term for various types of skin inflammation. As used herein it refers to eczema associated with abnormal or pathological expression of IL-31, over expression of IL-31, or eczema that is reduced in severity by administration of the saRNA disclosed herein. The saRNA and other products disclosed herein may be used to treat eczema, atopic dermatitis and other IL-3 mediated conditions.

Atopic dermatitis. A skin disease characterized by areas of severe itching, redness, scaling, and loss of the surface of the skin. Atopic dermatitis is the most common of the many types of eczema. Atopic dermatitis is frequently associated with other allergic disorders, such as asthma and hay fever. The saRNA and other products disclosed herein may be used to treat atopic dermatitis or other IL-31 related conditions.

Pruritus (itching). Uncomfortable irritation of the skin which results in scratching is a symptom of dermatological disease which may be defined as a cutaneous sensation provoking a desire to scratch or rub. The sensation of itch occurs as a result of activation of the cutaneous network of free nerve endings situated at the dermal epidermal junction. The saRNA and other products disclosed herein may be used to treat pruritus.

Canines, also called canids, include foxes, wolves, jackals, and other members of the dog family (Canidae). Canines include but are not limited to the dog or domestic dog (Canis familiaris or Canes lupus familiaris. The saRNAs disclosed herein can express canine IL-31, or epitopes, fragments, or variants of canine IL-31 or express IL-31 receptor molecules, or their epitopes, IL-31 contact residues, fragments, or variants.

Feline, (family Felidae), any of at least 37 cat species that among others include the cheetah, puma, jaguar, leopard, lion, lynx, tiger, and domestic cat. Felines include but are not limited to the domestic cat Felis catus. The saRNAs disclosed herein can express feline IL-31, or epitopes, fragments, or variants of feline IL-31 or express IL-31 receptor molecules, or their epitopes, IL-31 contact residues, fragments, or variants.

Equine (Equidae subfamily) is the taxonomic family of horses and related animals, including the extant horses, asses, donkeys, and zebras. All extant species are in the genus Equus, which originated in North America. Equines include but are not limited to the domestic horse Equus ferus caballus. The saRNAs disclosed herein can express equine IL-31, or epitopes, fragments, or variants of equine IL-31 or express IL-31 receptor molecules, or their epitopes, IL-31 contact residues, fragments, or variants.

Murine (subfamily Murinae) include Old and New World mice and rats in the family Muridae, which comprises at least 519 species. Murines include but are not limited to Mus musculus and other laboratory or inbred mice and albino Wistar, Sprague Dawley, Fischer 344, Holtzman albino strains, Long-Evans, and Lister black hooded rats and other laboratory or inbred strains of rats. The saRNAs disclosed herein can express murine IL-31, or epitopes, fragments, variants, or variants of murine IL-31 or express IL-31 receptor molecules, or their epitopes, IL-31 contact residues, fragments, variants, or variants.

The saRNA as disclosed herein may encode IL-31 its fragments or variants from the same or a different species. Advantageously, the saRNA will encode at least one IL-31 epitope that induces IL-31 neutralizing antibody or cellular responses. Thus, a saRNA that is administered to a canine may derive from canines expressing the same or different isoforms of IL-31 or derive from the IL-31 of a different species which comprises cross-reactive IL-31 epitopes with the IL-31 of the species to be vaccinated with the saRNA.

Alphavirus. The Alphavirus genus belongs to the Togaviridae family and contains 28 virus species (Griffin, 2007). Alphaviruses are positive-sense, single-stranded RNA viruses with a genome of approximately 11.5 kb in length. The positive sense genome contains two open reading frames (ORFs) and encodes four non-structural proteins and five structural proteins. The 5′ ORF encodes four non-structural proteins (nsP1-nsP4) and the 3′ ORF encodes the virus structural proteins (capsid and glycoproteins E3, E2, 6 K and E1). The non-structural proteins are translated from the positive-sense genomic RNA and function to transcribe full-length negative-sense RNA. Translation of the nsp1-3 polyprotein is terminated by an opal stop codon located between nsp3 and nsp4; the polyprotein nsp1-4 is produced when translational read through occurs at the nsp3-4 junction although there are notable exceptions where the opal stop codon is replaced by a sense codon, as is the case for strains of Semliki Forest virus (SFV), O'nyong-nyong virus (ONNV) and Sindbis virus (SIN). Features and elements of alphaviruses and alphavirus vectors are disclosed by, and incorporated by reference to Strauss & Strauss, The Alphaviruses: Gene Expression, Replication, and Evolution, MICROBIOL. REV. 1994, 58(3): 491-562.

Platform. This term covers constructs or vectors that can express an IL-31 polypeptide or antigen. It includes viral vector platforms such as a harmless viruses engineered to encode a polynucleotide encoding one or more antigens of interest and carry the polynucleotide into a cell, such as a canine cell. Such viral vectors may be replicating or non-replicating and cause a target cell to produce an IL-31 polypeptide, epitope, fragment, variant, or variant from the polynucleotide carried by the viral vector. This term also covers nucleic acid platforms incorporating messenger RNA that encodes one or more IL-31 antigens which are delivered to a target cell (e.g., a canine cell) often in a complex with a lipid or polymer-based nanoparticle. These also cause the target cell to produce an IL-31 antigen from the polynucleotide encoding the IL-31 antigen in the nucleic acid platform. Typically such an RNA platform is non-infectious as it can lack the structural protein sequences required to produce an infectious virus. This term also encompasses DNA platforms that likewise carry DNA encoding one or more IL-31 epitopes to be delivered to a target cell express the epitopes. Such DNA platforms may comprise a bacterial plasmid carrying the polynucleotide(s) encoding the IL-31 epitopes of interest.

Alphavirus platform. Alphavirus vectors have demonstrated high levels of transient heterologous gene expression both in vitro and in vivo and, therefore, possess attractive features for vaccine development. The most commonly used delivery vectors are based on three single-stranded encapsulated alphaviruses, namely Semliki Forest virus, Sindbis virus and Venezuelan equine encephalitis virus. An alphavirus platform that is advantageously used in many embodiments of technology disclosed herein encodes a polypeptide comprising at least one IL-31 epitope in place of alphavirus structural proteins. However, it contains the alphavirus replication sequences such as nsp1-nsp4 which can serve to amplify RNA encoding IL-31 antigens. In some embodiments, the alphavirus vectors may also be replication-deficient recombinant viral particles or as replication-proficient particles.

Non-limiting examples of modified non-alphavirus saRNAs or platforms include modified saRNAs of virus species belonging to Togaviridae family, Flaviviridae family, Orthomyxoviridae family, Rhabdoviridae family, or Paramyxoviridae family.

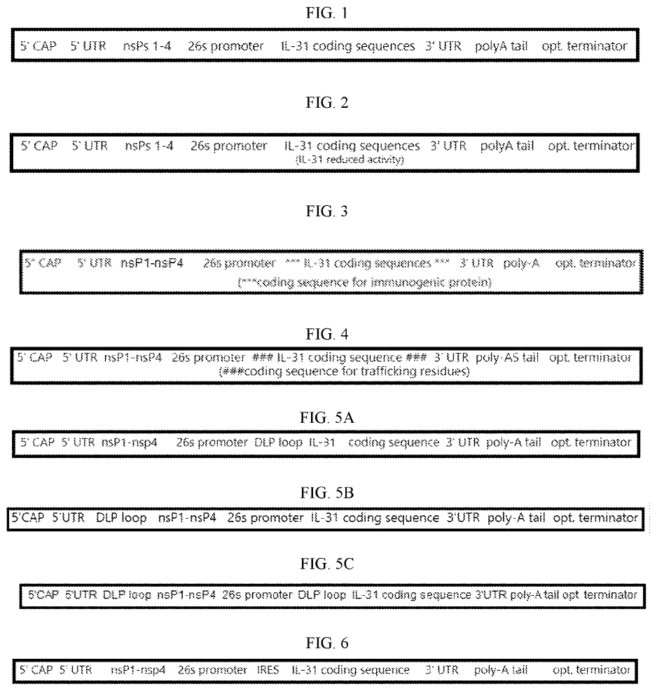

Self-amplifying RNA (saRNA). Self-amplifying RNA (saRNA) is a type of RNA vaccine derived from alphaviruses or flaviviruses and containing the viral replicase enzyme that allows it to amplify itself. It expresses the substituted genes (such as genes expressing IL-31 or its receptors) rather than those that encode the viral structural proteins. See Bloom, K. et al., Self-amplifying RNA vaccines for infectious diseases, GENE THERAPY, 202, 28, 117-129; Geall, A. J., Nonviral delivery of self-amplifying RNA vaccines, PNAS, 2012, 109(36), 14604-14609; Blakney, A. K., et al., An Update on Self-Amplifying mRNA Vaccine Development, VACCINES, 2021, 9(2), 97; each incorporated by reference. In a preferred embodiment, the saRNA as disclosed herein is based on Alphavirus architecture. Examples of saRNAs are described by FIGS. 1 to 6. saRNAs may be used as vectors to introduce RNA encoding protein antigens into a host cell, for example, for use as a vaccine.

The saRNA may include one or more of the following elements.

5′ Cap. The five-prime cap (5′ cap) is a specially altered nucleotide on the 5′ end of some primary transcripts such as precursor messenger RNA. This process, known as mRNA capping, is highly regulated and vital in the creation of stable and mature messenger RNA. Many preferred platforms as disclosed herein contain a 5′ cap. 5′ caps are well known. The alphavirus platforms as disclosed herein preferably have a 5′ cap.

5′ UTR. The 5′ untranslated region (also known as 5′ UTR, leader sequence, transcript leader, or leader RNA) is the region of a messenger RNA (mRNA) that is directly upstream from the initiation codon. This region is important for the regulation of translation of a transcript by differing mechanisms. A platform as disclosed herein usually contains a 5′UTR often encoding a leader peptide.

Alphavirus 26S promoter. The 26S promoter, which is located between the two ORFs on the alphavirus negative-sense RNA, is recognized by the alphavirus non-structural proteins for transcription of a sub-genomic mRNA from which structural proteins, such as exogenous IL-31 antigens, are translated. Various alphavirus 26S promoter sequences or their equivalents may be used in the platforms disclosed herein.

DLP loop. Downstream Hairpin Loop (DLP) are RNA structures that allow initiation of cap-dependent translation in the absence of the initiation factor eIF2, where the initiating methionine tRNA is placed on the ribosome by the DLP structure. CUG initiation involves initiation with a leucine and is more likely to occur under stress with eIF2 inhibition. Further description of DLPs, their structures, and uses are incorporated by reference to Kamrud, US 2018/0171340 A1.

IRES. An internal ribosome entry site, abbreviated IRES, is an RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. The location for IRES elements is often in the 5′ UTR but can also occur elsewhere in mRRNAs. In some alternative embodiments of the technology disclosed herein, the platform contains one or more IRES. An IRES may be omitted from many of the platforms disclosed herein as it is bulky and may decrease the uptake and transcription and translation efficiency of a platform containing it.

3′ UTR. The three prime untranslated region (3′ UTR) is the section of messenger RNA (mRNA) that immediately follows the translation termination codon. The 3′ UTR often contains regulatory regions that post-transcriptionally influence gene expression. A platform as disclosed herein usually contains a 3′UTR.

Poly-A. Polyadenylation is the addition of a poly(A) tail to an RNA transcript, typically a messenger RNA (mRNA). A poly(A) tail is added to an RNA at the end of transcription. On mRNAs, the poly(A) tail protects the mRNA molecule from enzymatic degradation in the cytoplasm and aids in transcription termination, export of the mRNA from the nucleus, and translation. The poly-A may be endogenous to the virus from which the platform is derived, such as endogenous to alphavirus or in other embodiments, may be exogenous.

Terminator. A transcription terminator is a section of nucleic acid sequence that marks the end of a gene or operon in genomic DNA during transcription. This sequence mediates transcriptional termination by providing signals in the newly synthesized transcript RNA that trigger processes which release the transcript RNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. A T7 terminator is one example of such a terminator. The T7 terminator is a sequence from bacteriophage T7 which allows efficient transcription termination. A T7 terminator or other functional terminators may be incorporated into a saRNA as described herein.

The term antigen as used herein and as commonly used in the field of immunology refers to an “antibody generating” molecule or a molecule that induces an antigen-specific T cells, i.e. a substance, which can elicit an adaptive immune response. An antigen is thus a molecule binding to an antigen-specific receptor, either a T-cell or a B-cell receptor. An antigen is usually a (poly)peptide, but it can also be a polysaccharide or a lipid, possibly combined with a protein or polysaccharide carrier molecule. For the purpose of the various aspects and embodiments of the present invention, the antigen is a polypeptide, i.e. an amino acid sequence. In the case of binding to a T-cell receptor, the antigen is presented to the respective T-cell receptor via an antigen-presenting cell as an antigenic peptide bound to a histocompatibility molecule on the surface of the antigen presenting cell, wherein the antigenic peptide has been processed in advance by the antigen presenting cell. Antigen presentation by professional antigen-presenting cells (APC) is the first step towards the initiation of an adaptive immune response carried out by naive T lymphocytes. Thus, an “antigen” as used herein refers to a molecule, such as a protein or a polypeptide, comprising one or more epitopes that will stimulate a host's immune system to make a humoral and/or cellular antigen-specific response.

The terms IL-31 immunogen or IL-31 receptor immunogen refer to all or part of the IL-31 or IL-31 receptor molecules capable of inducing an immune response, for example, a humoral or cellular response. Typically these immunogens contain T cell or B cell epitopes.

An epitope, also known as antigenic determinant, is the part of an antigen that is recognized by the immune system, specifically by antibodies, B cells, or T cells. In some embodiments, an IL-31, IL31RA, or Oncostatin M receptor β polypeptide or epitope may be glycosylated or post-translationally modified, e.g., by passage through the ER or Golgi.

The epitopes of protein antigens are divided into two categories, conformational epitopes and linear epitopes, based on their structure and interaction with the paratope. Conformational and linear epitopes interact with the paratope based on the 3-D conformation adopted by the epitope, which is determined by the surface features of the involved epitope residues and the shape or tertiary structure of other segments of the antigen. A conformational epitope is formed by the 3-D conformation adopted by the interaction of discontinuous amino acid residues. In contrast, a linear epitope is formed by the 3-D conformation adopted by the interaction of contiguous amino acid residues. T cell epitopes are presented on the surface of an antigen-presenting cell, where they are bound to major histocompatibility complex (MHC) molecules. In humans, professional antigen-presenting cells are specialized to present MHC class II peptides, whereas most nucleated somatic cells present MHC class I peptides. T cell epitopes presented by MHC class I molecules are typically peptides between 8 and 11 amino acids in length, whereas MHC class II molecules present longer peptides, 13-17 amino acids in length, and non-classical MHC molecules also present non-peptidic epitopes such as glycolipids.

Carrier proteins. Carrier proteins are generally chosen for their large size and distance from the host evolutionarily. The bigger the carrier and the more foreign in structure it is, the more immunogenic it is, increasing the chances of successfully developing antibodies. Each carrier has benefits over the other; the correct choice depends on the nature of the antigen of interest, the host being used to develop antibodies, and the final application for the antibody. Non-limited examples of carrier proteins are Keyhole limpet hemocyanin (KLH), Ovalbumin (OVA) and Bovine serum albumin (BSA). IL-31 and its fragments and variants may also be made more immunogenic by conjugation or admixture with other immunogens.

Lumazine Synthase. Lumazine synthase from Brucella spp. (BLS) is a highly immunogenic decameric protein which can accommodate foreign polypeptides or protein domains fused to its N-termini, markedly increasing their immunogenicity. In some embodiments, an SRV may include an insert encoding other types of lumazine synthase from bacteria, yeasts, or other organisms.

Breaking immune tolerance. Endogenous IL-31 is a self-protein and due to self-tolerance, in normal circumstances, is usually non-immunogenic. Tolerance to IL-31 may be broken by over-expressing IL-31, Immunizing with IL-31 and a immunogenic non-self-protein or with a fusion protein comprising non-self-amino acid residues and IL-31 residues, immunizing with denatured IL-31 for example by expression under conditions where proper folding does not occur, by immunizing with an altered variant of IL-31, for example, a IL-31 where one or more amino acid residues have been substituted with a non-natural amino acid at that residue, or by immunization with an exogenous form of IL-31, such as a different isoform than that expressed by a subject, or by immunization with xenogeneic IL-31. In the methods disclosed herein, one or more of these methods for breaking immune tolerance, or other known methods, may be employed. Immunizing as described above may comprise administering the saRNA disclosed herein under conditions which express denatured or modified IL-31 polypeptides, or express IL-31 polypeptides in combination with non-self-proteins or immunogenic carrier proteins.

IL-31 Neutralization. An epitope that induces an IL-31 neutralizing response may induce antibodies that inhibit or reduce IL-31 functional activity, such as its ability to cause pruritus or the other symptoms described herein. A neutralizing antibody or antigen-binding molecule may bind to IL-31 and block interactions between IL-31 and its receptors.

IL-31 receptor refers to either IL31RA or Oncostatin M receptor β, or to variants of these two proteins or to a complex IL31RA and Oncostatin M receptor β to which IL-31 can bind. Some variants do not functionally bind IL-31 or exhibit attenuated binding to IL-31 compared to natural forms. In some cases such as variants have at least 80, 85, 90, 95 or <100% sequence identity or similarity to the IL31RA or Oncostatin M receptor β molecules described herein.

IL31RA or Oncostatin M receptor β Neutralization. A neutralizing antibody to IL-31 or its receptor or other ligand that binds to IL-31 or its receptor may block interactions between IL-31 and its receptors, for example, by binding to, or sterically altering the configuration of, the contact residues on the receptor to which IL-31 binds. Interference with binding of IL-31 to its receptor blocks IL-31 signaling. IL31RA is related to gp130 (IL6ST; MIM 600694), the common receptor subunit for IL6 (MIM 147620)-type cytokines. Oncostatin M receptor (OSMR; MIM 601743) and IL31RA form the heterodimeric receptor through which IL31 (MIM 609509) signals.

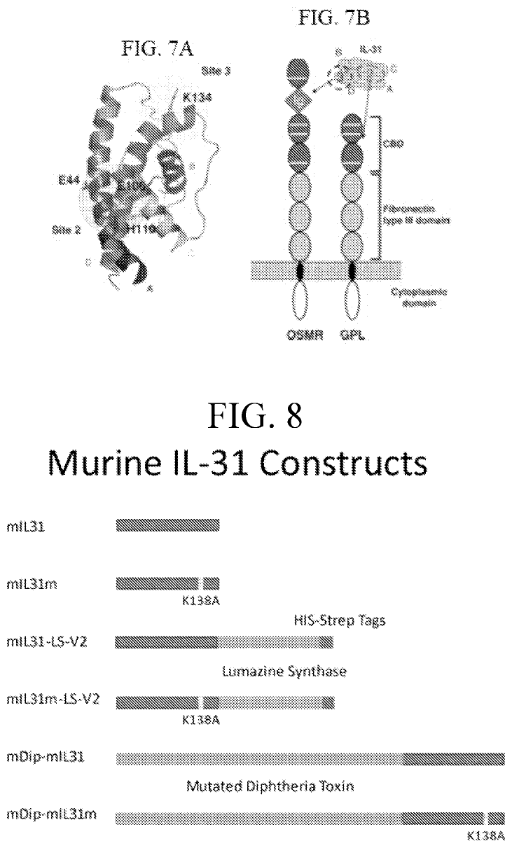

IL-31 is a 4-alpha helix bundle cytokine who's over expression is associated with pruritus; FIG. 7A. hIL-31 interacts with the cytokine binding domain of IL31RA via surface exposed residues on alpha helix A and C (site 2); FIG. 7B. It then recruits the oncostatin M receptor beta (OSMRβ) through alpha helix D (site 3). E44A, E106A, or H110A mutations in hIL-31 prevent binding to IL31RA while K134A was shown to inhibit the hIL-31-OMSR interaction. The functional outcome of these mutations is abrogation of IL-31 signaling. In some embodiments one, two, three, four, or more of the surface-exposed IL-31 residues interacting or binding to IL31RA are deleted or substituted so as to reduce or eliminate IL-31 activity or reduce the capacity of the mutated IL-31 to induce pruritis.

In some embodiments, the saRNA described herein induces antibodies that bind to IL-31 residues that contact an IL-31 receptor or that bind to contact residues of receptors recognized by IL-31. FIGS. 7A and 7B depict structures and key binding domains or residues of IL-31 and IL-31 receptors to which anti-IL-31 or anti-IL-31 receptor immune responses may be directed.

IL-31 can interact with the cytokine binding domain of IL31RA via surface exposed residues on the alpha helices A and C as shown for human IL-31 in FIGS. 7A and 7B. It then recruits the oncostatin M receptor beta (OSMRβ) through alpha helix D. In canine IL-31, the inventors modified homologous surface-exposed residues identified in human IL-31 to study interaction between IL-31 and its receptors. It is possible that the IL-31 receptor is not just one protein, but a series of interactions the produce functional ligand/receptor activation. Thus, targeting the IL31RA and/or OSMR components may modulate IL-31 activation and reduce the symptoms of AD.

A receptor antagonist is a type of receptor ligand or drug that blocks or dampens a biological response by binding to and blocking a receptor rather than activating it like an agonist. Antagonist drugs interfere in the natural operation of receptor proteins. Antibodies or peptides that bind to an IL-31 receptor can act as antagonists of IL-31 binding to the receptor.

Antigen processing is an immunological process that prepares antigens for presentation to special cells of the immune system called T lymphocytes. This process involves two distinct pathways for processing of antigens from an organism's own (self) proteins or intracellular pathogens (e.g., viruses), or from phagocytosed pathogens (e.g., bacteria); subsequent presentation of these antigens on class I or class II major histocompatibility complex (MHC) molecules is dependent on which pathway is used.

Variants of the polynucleotides or polypeptides disclosed herein may have a specific degree of sequence identity or similarity to said polynucleotides or polypeptides. BLASTN may be used to identify a polynucleotide sequence having at least 50%, 55%, 60%, 65%, 70%, 75%, 80%, 85%, 87.5%, 90%, 92.5%, 95%, 97.5%, 98%, 99% or <100% sequence identity to a reference polynucleotide, such as a polynucleotide encoding IL-31 or an IL-31 receptor or functional fragments thereof such as antigen coding sequences. A representative BLASTN setting modified to find highly similar sequences uses an Expect Threshold of 10 and a Wordsize of 28, max matches in query range of 0, match/mismatch scores of 1/-2, and linear gap cost. Low complexity regions may be filtered or masked. Default settings of a Standard Nucleotide BLAST are described by and incorporated by reference to <hypertext transfer protocol secure://blast.ncbi.nlm.nih.gov/Blast.cgi?PROGRAM=blastn&PAGE_TYPE=BlastSearch&LIN K_LOC=blasthome>(last accessed Jun. 7, 2022).

BLASTP can be used to identify an amino acid sequence having at least 50%, 55%, 60%, 65, 70%, 75%, 80%, 85%, 87.5%, 90%, 92.5%, 95%, 97.5%, 98%, 99% or <100% sequence identity, or similarity to a reference amino acid, such as a IL-31 or IL-31 receptor amino acid sequence, using a similarity matrix such as BLOSUM45, BLOSUM62 or BLOSUM80 where BLOSUM45 can be used for closely related sequences, BLOSUM62 for midrange sequences, and BLOSUM80 for more distantly related sequences. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity or similarity to the amino acid sequences disclosed herein are contemplated and encompassed by this disclosure. A representative BLASTP setting that uses an Expect Threshold of 10, a Word Size of 3, BLOSUM 62 as a matrix, and Gap Penalty of 11 (Existence) and 1 (Extension) and a conditional compositional score matrix adjustment. Other default settings for BLASTP are described by and incorporated by reference to the disclosure available at: <hypertext transfer protocol secure://blast.ncbi.nlm.nih.gov/Blast.cgi?PROGRAM=blastp&PAGE_TYPE=BlastSearch&LINK_LOC=blasthome>(last accessed Jun. 7, 2022).

Variants of a polynucleotide may include those with 1, 2, 3, 4, 5, 6, 7, 8, 9, 10, 20, 30 or more deletions, substitutions, or insertions of nucleotides into a polynucleotide such as those disclosed herein. Such variants may be based on a genomic virus sequence, on a sequence of a specific viral gene, or on sequences of other elements of a platform such as 3′ or 5′ UTR, IRES or any other sequence disclosed herein. In some cases, a variant polynucleotide sequence will be a naturally occurring variant of an IL-31 or IL-31 receptor sequence or an IL31RA or Oncostatin M receptor sequence.

Variants of polypeptides include those with 1, 2, 3, 4, 5, 6, 7, 8, 9, 10, 20, 30 or more deletions, substitutions, or insertions of amino acid residues into a polypeptide, such as those disclosed herein. Such variants may be based on one or more IL-31 polypeptides as disclosed herein or those of other mammals. In some cases, a variant amino acid sequence will be a naturally occurring variant of an IL-31 or IL-31 receptor sequence. Variants may also have post-translational modifications to one or more residues including glycosylation, chemical modification of N or C terminals, methylation, phosphorylation, ubiquitination, acetylation, hydroxylation, deamination, prenylation or partial proteolytic degradation.

Fragment or functional fragment as used herein refers to shorter or truncated segments of a longer nucleic acid or polypeptide sequence, preferably which retain at least one function of the whole nucleic acid or polypeptide, such as an epitope or a contact residue with another protein, such as contact residues on IL-31 or on IL-31 receptors. Functional immunogenic fragments are typically suitable as immunogens, as antigens, or as agonists or antagonists of binding between IL-31 and its receptor(s).

A fragment of a nucleic acid encoding an IL-31 antigen or immunogen, or IL-31 receptor antigen or immunogen, may encode at least one epitope of IL-31 or its receptors or at least one contact residue involved in the interaction of IL-31 with its receptor(s). Examples of polypeptide fragments include fragments of longer IL-31 polypeptides having at least 7, 8, 9, 10, 11, 12, 13, 14, 15, 16, 17, 18, 19, 20, 30, 40, 50 or more contiguous amino acid residues. Examples of polynucleotide fragments include those encoding the peptide fragments described above.

In some embodiments, an IL-31 fragment will consist or comprise a helical or non-helical domain of IL-31. In other embodiments, an IL-31 receptor will comprise a domain of IL31RA or Oncostatin M receptor or lack one or more domains of said receptors. For example, an IL-31 receptor may comprise, or lack, a cytoplasmic and/or transmembrane domain of IL31RA or Oncostatin M receptor or comprise or lack one or more fibronectin type III domains or CBD domains. In some embodiments, a IL31 receptor fragment will comprise domains that interact with IL-31, such as the Ig domain or OSMR or CBD domain of IL31RA.

In some embodiments, a fragment will comprise a domain, such as a cytoplasmic domain inside of a cell, a transmembrane domain, or an extracellular domain outside of a cell or a functional segment of such a domain. Such domains are described by accession numbers of the corresponding molecule or may be deduced by those skilled in the from the protein structure. Advantageously, portions of an extracellular domain may induce immune responses, such as cellular or humoral antibody responses, to external exposed portions of a molecule, such as IL-31 or its receptors.

Codon modification. In one embodiment, the one or more polynucleotides or other nucleic acid sequences of the platform or its polynucleotide components are codon modified based on the codon usage in the host cell or based on decreasing or increasing their GC content. Various functions and methods for codon modification may be used including those described by, and incorporated by reference to Hanson, G., Coller, J. Codon optimality, bias and usage in translation and mRNA decay. NAT REV MOL CELL BIOL 19, 20-30 (2018). <Hypertext transfer protocol secure://doi.org/10.1038/nrm.2017.91>; or <hypertext transfer protocol secure: //en.wikipedia.org/wiki/Codon_usage bias #Effect_on transcription_or_gene_expression>(last accessed Jun. 7, 2022) and by the references cited therein.

In another embodiment, the one or more polynucleotides or other nucleic acid sequences of the platform or its polynucleotide components are selected to attenuate formation of secondary RNA structures which reduce the expression of the at least one polynucleotide encoding IL-31 or at least one IL-31 receptor, their fragments or variants; provide or enhance secondary structures that stabilize the stability of mRNAs encoding IL-31 or IL-31 receptor antigens or immunogens; or to otherwise control the translation and relative abundance of multiple encoded IL-31 or IL-31 receptor immunogens or antigens.

In another embodiment, the one or more polynucleotides or other nucleic acid sequences of the platform or its polynucleotide components are selected to increase or decrease the expression of the at least one polynucleotide encoding an IL-31 antigen or immunogen or IL-31 receptor antigen or immunogen. For example, the codons encoding one IL-31 antigen may be selected to increase or decrease the relative abundance (or stoichiometric amount), of that antigen with respect to another IL-31 or IL-31 receptor immunogen or antigen.

Pharmaceutically acceptable carriers. A composition comprising the saRNA disclosed herein usually comprises a platform encoding a polypeptide having at least one IL-31 or IL-31 receptor epitope and at least one pharmaceutically acceptable carrier, excipient or diluent. Stabilizers or agents that prevent degradation of RNA may be included. Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate. In some embodiments, the carrier may be a particle, nanoparticle, liposome, lipoplex or lipid nanoparticle, for example, as described by and incorporated by reference to Kamrud, et al., US 2018/0171340 A.

LION. Lipid InOrganic Nanoparticle (LION). In some embodiments, the platform is administered as a nanoemulsion particle that has a hydrophobic core and comprises a mixture of a liquid oil and one or more inorganic solid nanoparticles. The nanoemulsion particle can also be referred to herein as Lipid InOrganic Nanoparticles (LIONs). The liquid oil is mixed with the one or more inorganic nanoparticles to form a hydrophobic core. The liquid oil is typically metabolizable. Suitable liquid oil can be a vegetable oil, animal oil, or synthetically prepared oil. In some embodiments, the liquid oil is a fish oil. In some embodiments, the liquid oil is a naturally occurring or synthetic terpenoid. In some embodiments, the liquid oil is squalene, triglyceride (such as capric/caprylic triglyceride or myristic acid triglyceride), vitamin E, lauroyl polyoxylglyceride, monoacylglycerol, soy lecithin, sunflower oil, soybean oil, olive oil, grapeseed oil, or a combination thereof. In one embodiment, the liquid oil is squalene, triglyceride (such as capric/caprylic triglyceride or myristic acid triglyceride), vitamin E, lauroyl polyoxylglyceride, monoacylglycerol, soy lecithin, or a combination thereof. In one embodiment, the liquid oil is squalene, triglyceride (such as capric/caprylic triglyceride or myristic acid triglyceride), sunflower oil, soybean oil, olive oil, grapeseed oil, or a combination thereof. In some embodiments, the liquid oil is squalene (either naturally occurring or synthetic, optionally in combination with any of the above listed liquid oils. The inorganic nanoparticles may be formed from one or more same or different metals (any metals including transition metal), such as from metal salts, metal oxides, metal hydroxides, and metal phosphates. Examples include silicon dioxide (SiO₂), iron oxides (Fe₃O₄, Fe₂O₃, FeO, or combinations thereof), aluminum oxide (A₁₂O₃), aluminum oxyhydroxide (AlO(OH)), aluminum hydroxyphosphate (Al(OH)ₓ(PO₄)_{y}), calcium phosphate (Ca₃(PO₄)₂), calcium hydroxyapatite (Ca₁₀(PO₄)_{·6}(OH)₂), iron gluconate, or iron sulfate. In some embodiments, the inorganic solid nanoparticle is a metal oxide, such as a transition metal oxide. In one embodiment, the inorganic solid nanoparticle is an iron oxide, for instance, magnetite (Fe₃O₄), maghemite (.gamma.-Fe₂O₃), wustite (FeO), hematite (α-Fe₂O₃), or combinations thereof. In some embodiments, the inorganic solid nanoparticle is a metal hydroxide, such as an aluminum hydroxide or aluminum oxyhydroxide. The inorganic solid nanoparticle may contain a reporter element detectable via imaging methods to allow for imaging and tracking the resulting nanoemulsion particles in the body. For instance, the inorganic solid nanoparticle may contain a reporter element detectable via magnetic resonance imaging (MRI), such as a paramagnetic, superparamagnetic, ferrimagnetic or ferromagnetic compound. Exemplary inorganic solid nanoparticle materials that are MRI-detectable are iron oxides, iron gluconates, and iron sulfates. The inorganic solid nanoparticle typically has an average diameter (number weighted average diameter) ranging from about 3 nm to about 50 nm. For instance, the inorganic solid nanoparticle can have an average diameter of about 5 nm, about 10 nm, about 15 nm, about 20 nm, about 25 nm, about 30 nm, about 35 nm, about 40 nm, about 45 nm, or about 50 nm. The inorganic solid nanoparticle may be surface modified before mixing with the liquid oil. For instance, if the surface of the inorganic solid nanoparticle is hydrophilic, the inorganic solid nanoparticle may be coated with hydrophobic molecules (or surfactants) to facilitate the miscibility of the inorganic solid nanoparticle with the liquid oil in the “oil” phase of the nanoemulsion particle. Phosphate-terminated lipids (such as phosphatidylated lipids), phosphorous-terminated surfactants, carboxylate-terminated surfactants, sulfate-terminated surfactants, or amine-terminated surfactants can be used for surface modification of the inorganic solid nanoparticle. Typical phosphate-terminated lipids or phosphorous-terminated surfactants are trioctylphosphine oxide (TOPO) or distearyl phosphatidic acid (DSPA). Typical sulfate-terminated surfactants include but not limited to sodium dodecyl sulfate (SDS). Typical carboxylate-terminated surfactants include oleic acid. Typical amine terminated surfactants include oleylamine. In one embodiment, the inorganic solid nanoparticle is a metal oxide such as an iron oxide, and a surfactant, such as oleic acid, oleylamine, SDS, DSPA, or TOPO, is used to coat the inorganic solid nanoparticle, before it is mixed with the liquid oil to form the hydrophobic core.] In one embodiment, the inorganic solid nanoparticle is a metal hydroxide, such as an aluminum hydroxide or aluminum oxyhydroxide, and a phosphate-terminated lipid or a surfactant, such as oleic acid, oleylamine, SDS, TOPO or DSPA is used to coat the inorganic solid nanoparticle, before it is mixed with the liquid oil to form the hydrophobic core. The lipids used to form nanoemulsion particles can be cationic lipids, anionic lipids, neutral lipids, or mixtures thereof. In some embodiments, the lipids used are cationic lipids. For example, positively charged lipids that can have favorable interactions with negatively charged bioactive agent (such as DNAs or RNAs) may be used in the nanoemulsion composition. Suitable cationic lipids include 1,2-dioleoyloxy-3-(trimethylammonium)propane (DOTAP); 3.beta.[N(N′,N′-dimethylaminoethane)-carbamoyl]cholesterol (DC Cholesterol); dimethyldioctadecylammonium (DDA); 1,2-dimyristoyl-3-trimethylammoniumpropane (DMTAP), dipalmitoyl(C16:0)trimethyl ammonium propane (DPTAP); distearoyltrimethylammonium propane (DSTAP); N-[1-(2,3-dioleyloxy)propyl]-N,N,N-trimethylammonium chloride (DOTMA); N,N-dioleoyl-N,N-dimethylammonium chloride (DODAC); 1,2-dioleoyl-sn-glycero-3-ethylphosphocholine (DOEPC); 1,2-dioleoyl-3-dimethylammonium-propane (DODAP); and 1,2-dilinoleyloxy-3-dimethylaminopropane (DLinDMA); 1,1′-((2-(4-(2-((2-(bis(2-hydroxydodecyl)amino)ethyl) (2-hydroxydodecyl)amino)ethyl)piperazin-1-yl)ethyl)azanediyl)bis(dodecan-2-ol) (C12-200); and combinations thereof. A typical cationic lipid is DOTAP. Other examples for suitable lipids include, but are not limited to, the phosphatidylcholines (PCs), such as distearoylphosphatidylcholine (DSPC), dioleoyl phosphatidylcholine (DOPC), 1-palmitoyl-2-oleoylphosphatidylcholine (POPC), dipalmitoylphosphatidylcholine (DPPC), dipalmitoylphosphatidylcholine (DMPC), etc; phosphatidylethanolamines (PEs), such as 1,2-distearoyl-sn-glycero-3-phosphoethanolamine (DSPE), dioleoylphosphatidylethanolamine (DOPE), etc.; phosphatidylglycerol (PGs); and PEGylated lipids including PEGylated version of any of the above lipids (e.g., DSPE-PEGs). The nanoemulsion particle can further contain one or more surfactants, which can be a hydrophobic surfactant or a hydrophilic surfactant. In some embodiments, the nanoemulsion particle further comprises a hydrophobic surfactant. In some embodiments, the nanoemulsion particle further comprises a hydrophilic surfactant. In one embodiment, the nanoemulsion particle further comprises a hydrophobic surfactant and a hydrophilic surfactant. Suitable hydrophobic surfactants include those having a hydrophilic-lipophilic balance (HLB) value of 10 or less, for instance, 5 or less, from 1 to 5, or from 4 to 5. An exemplary hydrophobic surfactant is a sorbitan ester (such as sorbitan monoester or sorbitan trimester). For instance, the hydrophobic surfactant can be a sorbitan ester having a HLB value from 1 to 5, or from 4 to 5. In some embodiments, the hydrophobic surfactant is a sorbitan monoester or a sorbitan triester. Exemplary sorbitan monoesters include sorbitan monostearate and sorbitan monooleate. Exemplary sorbitan triesters include sorbitan tristearate and sorbitan trioleate. Suitable hydrophilic surfactants include those polyethylene oxide-based surfactants, for instance, a polyoxyethylene sorbitan ester (polysorbate). In some embodiments, the hydrophilic surfactant is a polysorbate. Exemplary polysorbates are polysorbate 80 (polyoxyethylene sorbitan monooleate, or Tween 80), polysorbate 60 (polyoxyethylene sorbitan monostearate, or Tween 60), polysorbate 40 (polyoxyethylene sorbitan monopalmitate, or Tween 40), and polysorbate 20 (polyoxyethylene sorbitan monolaurate, or Tween 20). In one embodiment, the hydrophilic surfactant is polysorbate 80. The nanoemulsion particle can have an oil-to-surfactant molar ratio ranging from about 0.1:1 to about 20:1, from about 0.5:1 to about 12:1, from about 0.5:1 to about 9:1, from about 0.5:1 to about 5:1, from about 0.5:1 to about 3:1, or from about 0.5:1 to about 1:1. The nanoemulsion particle can have a hydrophilic surfactant-to-lipid (e.g., cationic lipid) ratio ranging from about 0.1:1 to about 2:1, from about 0.2:1 to about 1.5:1, from about 0.3:1 to about 1:1, from about 0.5:1 to about 1:1, or from about 0.6:1 to about 1:1. The nanoemulsion particle can have a hydrophobic surfactant-to-lipid (e.g., cationic lipid) ratio ranging from about 0.1:1 to about 5:1, from about 0.2:1 to about 3:1, from about 0.3:1 to about 2:1, from about 0.5:1 to about 2:1, or from about 1:1 to about 2:1. The nanoemulsion particle can comprise from about 0.2% to about 40% w/v liquid oil, from about 0.001% to about 10% w/v inorganic solid nanoparticle, from about 0.2% to about 10% w/v lipid (e.g., cationic lipid), from about 0.25% to about 5% w/v hydrophobic surfactant (e.g., sorbitan ester), and from about 0.5% to about 10% w/v hydrophilic surfactant. In certain embodiments, the nanoemulsion particle comprises: a hydrophobic core comprising a mixture of one or more inorganic nanoparticles containing at least one metal oxide nanoparticle optionally coated with a phosphate-terminated lipid, a phosphorous-terminated surfactant, a carboxylate-terminated surfactant, a sulfate-terminated surfactant, or an amine-terminated surfactant, and a liquid oil containing naturally occurring or synthetic squalene; a cationic lipid comprising DOTAP; a hydrophobic surfactant comprising a sorbitan ester selected from the group consisting of sorbitan monostearate, sorbitan monooleate, and sorbitan trioleate; and a hydrophilic surfactant comprising a polysorbate. In one embodiment, the nanoemulsion particle comprises: a hydrophobic core comprising a mixture of: one or more inorganic nanoparticles containing iron oxide nanoparticles, and a liquid oil containing naturally occurring or synthetic squalene; the cationic lipid DOTAP; a hydrophobic surfactant comprising sorbitan monostearate; and a hydrophilic surfactant comprising polysorbate 80. In this LION composition, the LION particle can comprise from about 0.2% to about 40% w/v squalene, from about 0.001% to about 10% w/v iron oxide nanoparticles, from about 0.2% to about 10% w/v DOTAP, from about 0.25% to about 5% w/v sorbitan monostearate, and from about 0.5% to about 10% w/v polysorbate 80. In one embodiment, the LION particle comprises from about 2% to about 6% w/v squalene, from about 0.01% to about 1% w/v iron oxide nanoparticles, from about 0.2% to about 1% w/v DOTAP, from about 0.25% to about 1% w/v sorbitan monostearate, and from about 0.5%) to about 5% w/v polysorbate 80. In certain embodiments, the nanoemulsion particle comprises: a hydrophobic core comprising a mixture of: one or more inorganic nanoparticles containing at least one metal hydroxide or oxyhydroxide nanoparticle optionally coated with a phosphate-terminated lipid, a phosphorous-terminated surfactant, a carboxylate-terminated surfactant, a sulfate-terminated surfactant, or an amine-terminated surfactant, and a liquid oil containing naturally occurring or synthetic squalene; a cationic lipid comprising DOTAP; a hydrophobic surfactant comprising a sorbitan ester selected from the group consisting of sorbitan monostearate, sorbitan monooleate, and sorbitan trioleate; and a hydrophilic surfactant comprising a polysorbate. In one embodiment, the nanoemulsion particle comprises: a hydrophobic core comprising a mixture of one or more inorganic nanoparticles containing aluminum hydroxide or aluminum oxyhydroxide nanoparticles optionally coated with TOPO, and a liquid oil containing naturally occurring or synthetic squalene; the cationic lipid DOTAP; a hydrophobic surfactant comprising sorbitan monostearate; and a hydrophilic surfactant comprising polysorbate 80. In this LION composition, the LION particle can comprise from about 0.2% to about 40% w/v squalene, from about 0.001% to about 10% w/v aluminum hydroxide or aluminum oxyhydroxide nanoparticles, from about 0.2% to about 10% w/v DOTAP, from about 0.25% to about 5% w/v sorbitan monostearate, and from about 0.5% to about 10% w/v polysorbate 80. In one embodiment, the LION particle comprises from about 2% to about 6% w/v squalene, from about 0.01% to about 1% w/v aluminum hydroxide or aluminum oxyhydroxide nanoparticles, from about 0.2% to about 1% w/v DOTAP, from about 0.25% to about 1% w/v sorbitan monostearate, and from about 0.5%) to about 5% w/v polysorbate 80. Nanoparticles and nanoemulsions have been described in the literature and the terms are used herein to refer to those particles having a size less than 1000 nanometers. The nanoemulsion particle (LION) typically has an average diameter (z-average hydrodynamic diameter, measured by dynamic light scattering) ranging from about 20 nm to about 200 nm. In some embodiments, the z-average diameter of the LION particle ranges from about 20 nm to about 150 nm, from about 20 nm to about 100 nm, from about 20 nm to about 80 nm, from about 20 nm to about 60 nm. In some embodiments, the z-average diameter of the LION particle ranges from about 40 nm to about 200 nm, from about 40 nm to about 150 nm, from about 40 nm to about 100 nm, from about 40 nm to about 90 nm, from about 40 nm to about 80 nm, or from about 40 nm to about 60 nm. In one embodiment, the z-average diameter of the LION particle is from about 40 nm to about 80 nm. In one embodiment, the z-average diameter of the LION particle is from about 40 nm to about 60 nm. The average polydispersity index (PDI) of the nanoemulsion particles (LIONs) can range from about 0.1 to about 0.5. For instance, the average PDI of the LION particles can range from about 0.2 to about 0.5, from about 0.1 to about 0.4, from about 0.2 to about 0.4, from about 0.2 to about 0.3, or from about 0.1 to about 0.3.

Modes of administration. In some embodiments, the polynucleotides or compositions disclosed herein are formulated for in vivo delivery. For administration to an animal, the composition according to the present application can be formulated for, or given by, any enteral or parenteral route. These include oral, mucosal, bucally, rectally, optically, nasal or respiratory, by inhalation, by spray, by aerosol, topical, intradermal, intralesional, subcutaneous, intramuscular, intravenous, intraorgan, into a sex organ, intra-articularly, intra-synovially, intrasternally, intrathecally, intrahepatically, intrathymically, intracranially, or intraventricularly. The saRNA may be administered directly or by use of a reservoir that provides release over a period of time.

Oral/Mucosal Administration. In one embodiment, the saRNA as disclosed herein may be administered orally or mucosally. The delivery of vaccines through an oral or mucosal route can elicit a potent mucosal response considering the extensive presence of gut-associated lymphoid tissues (GALT). For example, DNA encoding the saRNA disclosed herein may be administered to cells, for example, via transgenic Salmonella typhimurium, Lawsonia intracellularlris, or other intracellular bacteria that invade host cells, release the plasmid encoding the saRNA, which is then transcribed into the saRNA, which expresses IL-31, its fragments or variants, or IL-31 receptor components, fragments, variants or variants; see Jawalagatti, V., et al., Oral mRNA Vaccines Against Infectious Diseases—A Bacterial Perspective, FRONT. IMMUNOL. 2022, 13:884862, incorporated by reference.

This method provides an easy way to orally or mucosally administer the saRNA to a subject animal without the need for injection. In another embodiment DNA encoding the saRNA may be incorporated into or transfected into a cell by other means known in the art including by electroporation, viral transduction, endocytosis of particles containing the DNA, by injection of particles, such as calcium phosphate particles containing the DNA using a gene gun. Cells may be transfected in vivo, ex vivo or in vitro. In the latter cases, they may be reintroduced into a subject animal to provoke immune responses against IL-31, its fragments or variants.

### Embodiments

One aspect of this technology is directed to a saRNA (self-amplifying RNA) that encodes IL-31 or an IL-31 variant, or an immunogenic fragment or epitope thereof; and/or which encodes an IL-31 receptor or receptor variant, fragment or epitope thereof, which comprises in any order:
(a) a first nucleic acid sequence comprising nsP1, nsP2, nsP3 and nsP4 that encode non-structural proteins of the alphavirus, and
(b) a second nucleic acid sequence that encodes IL-31, the IL-31 variant, or immunogenic fragment or epitope thereof; and/or a second nucleic acid sequence that encodes an IL-31 receptor or its subunits or domains, or variants, fragments or epitopes thereof; and
(c) wherein the saRNA comprises a 5′ cap, a 5′ UTR upstream of the first nucleic acid sequence, a 26S promoter upstream of the second nucleic acid sequence, a 3′UTR, and a poly A tail;
which saRNA when incorporated into a cell expresses IL-31, the IL-31 variant, or the immunogenic fragment or epitope thereof; or expresses an IL-31 receptor, receptor variants, receptor subunits, domains, its fragments, or its epitopes. In some embodiments, a fragment of IL-31 or an IL-31 receptor will comprise contact residues for an IL-31 receptor or IL-31, respectively and may act as an antagonist by binding to IL-31 receptor and blocking binding of IL-31; or by binding to IL-31 and blocking it from binding to its receptor.

In an alternative embodiment, the saRNA disclosed herein is encoded by a DNA vector or DNA construct from which the saRNA is transcribed.

One aspect of the saRNA disclosed above contains an alphavirus scaffold obtained from an Old World alphavirus selected from the group consisting of Sindbis virus (SINV), Chickungunya virus (CHIKV), Semliki Forest Virus (SFV), Ross River Virus (RRV), Sagiyama virus (SAGV), Getah virus (GETV), Middleburg virus (MIDV), Bebaru virus (BEBV), O'nyong nyong virus (ONNV), Ndumu (NDUV), and Barmah Forest virus (BFV); or from a New World alphavirus selected from the group consisting of Venezuelan Equine Encephalitis Virus (VEEV), western equine encephalitis virus (WEEV), and eastern equine encephalitis virus (EEEV

RNA encoding IL-31 or its variants, fragments, or epitopes, or RNA encoding an IL-31 receptor, its subunits, variants, fragments or epitopes, is operably incorporated into such a scaffold to express these proteins. Typically, the exogenous RNA replaces the structural genes of the alphavirus during construction of the saRNA.

In another embodiment of this technology, the saRNA described herein comprises, consists essentially of, or consists of, in the following order the 5′ cap, the 5′ UTR, nsP1, nsP2, nsP3, nsP4, the 26S promoter, the second nucleic acid sequence, the 3′UTR and the poly A tail. In some embodiments, a terminator sequence may be present at the end of a gene or polynucleotide sequence encoding IL-31 or its fragments or variants (or IL-31 receptor or its subunits, fragments or variants) facilitating release of the transcriptional complex which can free RNA polymerase and related transcriptional machinery to begin transcription of new mRNAs.

In some embodiments, the second nucleic acid sequence of the saRNA will encode a wild-type canine, murine, equine or feline IL-31 or IL-31 receptor as described by the accession numbers disclosed herein.

In other embodiments, the second nucleic acid sequence of the saRNA encodes an IL-31 variant or IL-31 receptor variant containing 1, 2, 3, 4, 5, 6, 7, 8, 9 or 10 insertions, substitutions, or deletions to the wild-type sequence of canine IL-31 (NP_001159386.1), murine IL-31 (AAS82846.1), equine IL-31 (XP_023503836.1), or feline IL-31 (XP_044897990.1); or wherein said variant has at least 50, 60, 70, 80, 90, 95, 99. 99.5, 99.9, or <100% sequence identity to the wild-type sequence of canine IL-31 (NP_001159386.1), murine IL-31 (AAS82846.1), equine IL-31 (XP_023503836.1), or feline IL-31 (XP_044897990.1); or by the other accession numbers disclosed herein or in the GENBANK or UNIPROT databases; or wherein said variant is a homolog or isoform of the IL-31 of sequences identified by the accession numbers above or other accession numbers disclosed herein.

In other embodiments, the second nucleic acid sequence of the saRNA encodes an IL-31 receptor variant containing 1, 2, 3, 4, 5, 6, 7, 8, 9 or 10 insertions, substitutions, or deletions to the wild-type sequence of IL-31 or its subunits; or wherein said variant has at least 50, 60, 70, 80, 90, 95, 99. 99.5, 99.9, or <100% sequence identity to the wild-type sequence of IL-31 receptor or its subunits.

IL-31 variants or IL-31 receptor variants may be produced by site-directed mutagenesis as described by Le Sauz, et al., Molecular dissection of human interleukin-31 mediated signal transduction through site-directed mutagenesis, J. BIOL. CHEM. 2010, 285(5):3470-99 (incorporated by reference). Critical ligand-binding determinants of IL-31 and other conserved IL-31 residues are incorporated by reference to Le Sauz, et al., id.

Corresponding ligand binding residues in IL-31 of other species may be identified from those described by Le Sauz, supra who reports that E44A, E106A, or H110A mutations in hIL-31 prevented binding to IL31RA while K134A was shown to inhibit the hIL-31-OMSR interaction. The functional outcome of these mutations is abrogation of IL-31 signaling.

Mutation of corresponding or adjacent residues of the non-human IL-31 is disclosed herein may have the same effect as mutation of these residues in human IL-31 as based on NCBI Reference Sequence: NP_001014358.1. For example, the corresponding or adjacent canine residues to human based on canine IL-31 accession number NP_001159386.1 are for E44A (canine 42-44), for E106A (canine 105-107), for H110A (canine 108-110) and for K134A (canine 132-134). Based on local similarity between human and canine IL-31 sequences, substitution of one or more canine IL-31 residues 42-44, 105-107, 108-110, or 132-134 with alanine or another heterologous residue may also inhibit IL-31 interaction with its receptors.

In other embodiments the second nucleic acid sequence encodes a variant of wild-type canine, murine, equine, or feline IL-31 or wild-type IL-31 receptor(s) as described by the accession numbers disclosed herein, wherein said variant has 1, 2, 5, 10, 20, 30, 40, 50, 60, 70, 80, 90, 100 or >100% less IL-31 activity than the corresponding wild-type IL-31 or 1, 2, 5, 10, 20, 30, 40, 50, 60, 70, 80, 90, 100 or >100% less IL-31 binding or signal transducing activity than the corresponding wild-type receptor. Modifications may be made to IL-31 to reduce its activity and ability to induce atopic dermatitis while retaining immunogenic epitopes. Modifications may be made to IL-31 receptors to enhance their ability to antagonize IL-31 to its native receptors, for example, by producing soluble IL-31 receptor forms that antagonize IL-31 binding to its native receptor, for example, by designing peptides that bind to IL-31 contact residues or removing transmembrane or cytoplasmic residues or domains to render a receptor soluble so that it can nonproductively bind circulating IL-31.

In some embodiments, the second nucleic acid sequence encodes a variant of canine IL-31 comprising K133A, a variant of murine IL-31 comprising K138A, or a variant of equine IL-31 comprising K137A; or wherein the K at an aforementioned residue K133, K138 or K137 is replaced by an amino acid other than K. In other embodiments, the second nucleic acid may encode variants having substitutions to IL-31 contact residues for its receptor(s) such as those described herein for canine IL-31.

In other embodiments, the second nucleic acid sequence encodes a variant of canine IL-31 having a single substitutional mutation K133A; a variant of murine IL-31 having a single substitutional mutation K138A; a variant of equine IL-31 having a single substitutional mutation K137A; or a variant or variant of the accession numbers disclosed herein, respectively, having a single substitutional mutation at K133, K138 or K137 which replaces said residue with an amino acid other than K.

In some embodiments, the second nucleic acid sequence encodes IL-31, an IL-31 variant, or a fragment or epitope thereof that binds to a cytokine binding domain of (IL31RA) and/or to oncostatin M receptor beta (OSMRp) or to a complex of these two subunits.

In certain embodiments, the second nucleic acid sequence encodes IL-31, an IL-31 variant, or a fragment or epitope thereof that does not bind to the cytokine binding domain of IL31RA and/or to oncostatin M receptor beta (OSMRp). Such fragments or variants lack the ability to bind to the IL-31 receptor or its components and deliver or transduce a signal via binding but can maintain immunogenic epitopes capable of inducing anti-IL-31 immune responses.

In particular embodiments, the saRNA disclosed herein further comprises a nucleic acid sequence encoding an immunogenic carrier, wherein the nucleic acid sequence encoding the immunogenic carrier is optionally fused to the second nucleic acid sequence in the same open reading frame and expresses a fusion protein comprising IL-31, the IL-31 variant, or immunogenic fragments or epitopes thereof, and the immunogenic carrier; or expresses a fusion protein comprising IL-31 receptor, an IL-31 receptor variant, or immunogenic fragments or epitopes thereof, and the immunogenic carrier.

Such a carrier may comprise a genetically modified cross-reacting material (CRM) of diphtheria toxin, tetanus toxoid (T), meningococcal outer membrane protein complex (OMPC), diphtheria toxoid (D), or H. influenzae protein D (HiD), wherein the subsequent nucleic acid sequence is optionally fused. In preferred embodiments, the carrier may be an immunogenic portion of Lumazine synthase.

In other embodiments, the saRNA disclosed herein further comprises a nucleic acid sequence encoding, at least one immunomodulatory protein including but not limited to chemokines CXCL-8, CCL2, CCL3, CCL4, CCL5, CCL11, CXCL10, CCL14, CCL19, CCL20, CCL21, CCL25, CCL27, CXCL12, or CXCL13; interleukins IL-1, IL-2, IL-3, IL-4, IL-5, IL-6, IL-7, IL-8, IL-9, IL-10, IL-11, IL-12, IL-12A, IL-13, IL-14, IL-15, IL-17, or IL-18; cytokines colony stimulating factor 1 (CSF1), colony stimulating factor 2 (CSF2), or colony-stimulating factor 3 (CSF 3); proteins that an intracellular pattern recognition receptor (PRR): Sendai virus-derived oligonucleotide that imitates Sendai virus defective interfering (DI) particles (SeVDI) or another PRR triggering protein; or combinations thereof. An immunomodulatory protein may be expressed fused to an IL-31 protein or independently.

In some embodiments, the saRNA disclosed herein further comprises at least one viral capsid enhancer or downstream loop (“DLP”) and may also comprise a protein separation site, ribosome stalling site, or protease cleavage site between the at least one viral capsid enhancer or DLP and the second nucleic acid sequence.

In other particular embodiments, the saRNA disclosed herein may further contain at least one internal ribosome entry site (IRES) that is upstream of the second nucleic acid sequence encoding IL-31, its variants, fragments or epitopes or encoding IL-31 receptor, its subunits, domains, variants, fragments or epitopes.

Another aspect of this technology is directed to a plasmid or other DNA construct encoding the saRNA disclosed herein. Such a plasmid or DNA construct may be incorporated into a cell, such as cell capable of expressing nucleic acid sequences encoding IL-31, its fragments and variants or encoding IL-31 receptor, its subunits, domains, variants, fragments or epitopes.

Another aspect of the technology is a composition comprising the saRNA disclosed herein in combination with a pharmaceutically acceptable carrier or excipient. The pharmaceutically acceptable carrier or excipient may comprise at least one of lipids, surfactants, polyamino acids, proteins, polymeric particles, self-assembled particles, composite nanoparticles of conjugated phospholipids, or inorganic particles (such as silica particles), or combinations thereof.

In another embodiment, the composition comprises lipid inorganic nanoparticles (LION).

The composition disclosed above may be formulated for administration by a variety of different routes include, but not limited to, routes for oral or mucosal administration; for intravenous, intramuscular or other parenteral administration.

The composition disclosed above may be in a particulate or nanoparticulate form; in the form of a liposome, lipoplex, or lipid nanoparticle (LNP); or in the form of a nanoemulsion comprising a hydrophobic core that comprises a mixture of a liquid oil and one or more inorganic nanoparticles, one or more lipids and, optionally, one or more surfactants.

A practical aspect of this technology is a method for reducing the severity of atopic dermatitis in a mammal comprising, consisting essentially of, or consisting of, administering to a subject in need thereof an saRNA that encodes IL-31, an IL-31 variant thereof, or an immunogenic fragment or epitope thereof. In other embodiments, the method involves administering an saRNA that encodes IL-31 receptor, an IL-31 receptor variant, its subunits, its domains, its fragments or epitopes. As used herein “IL-31 receptor” encompasses IL31RA or its variants, Oncostatin M beta receptor or its variants, as well as a complexes comprising both of these protein subunits.

In some embodiments, a plasmid or DNA construct encoding IL-31 or IL-31 receptor antigens, a cell expressing IL-31 or IL-31 receptor polypeptides, or a composition containing these embodiments may be administered.

In the method disclosed above, the mammal may be a canine and the IL-31, IL-31 variant or immunogenic fragments thereof are derived from canine IL-31; or the mammal may be a murine, equine, or feline and the IL-31, IL-31 variant or immunogenic fragments thereof are derived from murine, equine or feline IL-31.

In the method disclosed above, the mammal may be a canine and the IL-31 receptor, IL-31 receptor variant or immunogenic or IL-31 antagonist fragments are derived from canine IL-31 receptor; or the mammal may be a murine, equine, or feline and the IL-31 receptor, IL-31 receptor variant or immunogenic or IL-31 antagonist fragments thereof are derived from murine, equine or feline IL-31. In some embodiments, saRNA(s) encoding IL-31 or its variants and IL-31 receptors or their variants may be administered.

The method disclosed above may be directed toward treatment or reduction of severity of atopic dermatitis in a subject in an early phase characterized by pruritus and by abnormal scratching and/or licking, and/or by infiltration of TH2 lymphocytes upon examination of biopsied skin, but with normal looking skin with substantially no redness.

The method disclosed above may be directed toward treatment or reduction of severity of atopic dermatitis in a subject where the atopic dermatitis is in an acute phase characterized by abnormal skin redness and skin excoriation due to scratching and/or by comorbid infections.

The method disclosed above may be directed toward treatment or reduction of severity of atopic dermatitis in a subject where the atopic dermatitis is in a chronic stage characterized by thickening of the skin, lichenification, microbial infection, and/or darkening of affected skin.

The method disclosed above may be performed by administering the saRNA by various routes including orally or mucosally; subcutaneously, intramuscularly, intravenously, or parenterally. In one embodiment, a priming dose of the saRNA is administered, followed several weeks or months later by a booster dose, and then subsequently with an annual booster dose.

The method may further comprise administering at least one anti-inflammatory drug or antihistamine drug, such as, but not limited to an anti-inflammatory drug selected from a glucocorticoid, a calcineurin Inhibitor, a phosphodiesterase 4 (PDE4) inhibitor, a protease activated receptor (PAR) inhibitor, a periostin inhibitor or recombinant interferon gamma.

This method may further comprise administering at least a monoclonal antibody directed to IgE or to a cytokine selected from the group consisting of IL2, IL4, IL5, IL6, IL8, IL9, IL13, IL18, IL22, IL-31, IL33, CCL4, TSLP, OX40L or oncostatin M, or at least a monoclonal directed to a receptor of said cytokine.

This method may further comprise administering at least one soluble receptor of IgE or a soluble receptor of a cytokine selected from the group consisting of IL2, IL4, IL5, IL6, IL8, IL9, IL13, IL18, IL22, IL-31, IL33, CCL4, TSLP, OX40L or oncostatin M, or an extracellular domain of said receptor fused with a Fc fragment.

This method may further comprise administering kinase inhibitor including but not limited to a JAK inhibitor such as oclacitinib, a kinase inhibitor such as LLRK2, IRAK, or SYK, or an allergen-specific immunotherapy (ASIT).

In some embodiments of this method the saRNA is administered in a nanoemulsion comprising a hydrophobic core that comprises a mixture of a liquid oil and one or more inorganic nanoparticles, one or more lipids and, optionally, one or more surfactants.

Another aspect of the invention is directed to a method for making or constructing the saRNA disclosed herein, comprising operably inserting into an saRNA a nucleic acid sequence that expresses IL-31 or an immunogenic fragment or epitope thereof, or a variant thereof; or which expresses an IL-31 receptor, receptor variant, or fragment thereof. This method of making may further comprise operably inserting at least one nucleic acid sequence encoding a 5′ cap, a 5′ UTR upstream of the first nucleic acid sequence, a 26S promoter upstream of the second nucleic acid sequence, a 3′UTR, a poly A tail; a protease, a protease cleavage site, a ribosome stalling site or protein separation site, a DLP loop, an IRES, an immunogenic carrier, an immunomodulatory protein, or a cytokine.

In other aspects of this method the IL-31 fragment or IL-31 variant has less IL-31 activity than a corresponding IL-31 expressed by a murine, canine, equine, or feline cell; or wherein the IL-31 fragment or variant induces less pruritus or fewer or less severe dermatological symptoms when administered to said murine, canine, equine, or feline.

In other aspects of this method IL-31 or IL-31 receptor expressed by the saRNA is folded differently or is post-translationally or epigenetically modified compared to IL-31 or IL-31 receptor produced by an untransformed murine, canine, equine or feline cell; or wherein the IL-31 or IL-31 receptor is expressed in the cytoplasm or expressed via the endoplasmic reticulum and Golgi apparatus.

To produce the second nucleic acid sequence of the saRNA, one may replace at least one degenerate codon in the nucleic acid sequence which encodes IL-31 or IL-31 receptor or an immunogenic fragment or variant thereof, with a different codon encoding the same amino acid, which different codon increases GC content, reduces RNA hairpin formation, increases expression of IL-31, IL-31 fragments or IL-31 variants, increases expression of IL-31 receptor components, or increases the stability of the saRNA.

In another aspect of this method of making, the saRNA comprises a self-amplifying RNA engineered for resistance to cellular innate immune responses.

### Example 1. Construction of Murine IL-31 SRV

Design of 6 mIL-31 Genes of Interest. As depicted by FIG. 8 below, two versions of the murine IL-31 gene of interest (GOI) are designed in silico, a native version (mIL-31) and an inactivated version with the K138A mutation (mIL-31m). These two IL-31 open reading frames (ORF) are also used to create 4 other GOIs. Two of these GOIs include fusing the 3′ ends of each IL-31 ORF (native and inactivated independently) to the 5′ end of the Lumazine Synthase ORF. These clones contain 6×His and Strep tags on the 3′ end of the gene. The final two GOIs include fusing the 3′ end of the inactivated Diphtheria toxin ORF to the 5′ end of each IL-31 ORF (native and inactivated independently) with the IL-31 signal peptide removed; see FIG. 8.

The 5′ and 3′ ends of all 6 GOI are modified to contain additional nucleotides to aid in cloning into the replicon. On the 5′ end, the EcoRV restriction endonuclease site (GATATC) and Kozak sequence (GCCACC) are added upstream of the IL-31 or Diphtheria toxin start codon. On the 3′ end, the GCTGC sequence and PacI restriction endonuclease site (TTAATTAA) are added downstream of the IL-31 or Lumazine Synthase stop codon. All 6 GOIs are ordered as double stranded DNA fragments called gBlock™ from Integrated DNA Technologies.

Ligation of IL-31 Genes of Interest into VEE backbone. The pSGV_023_KAN Alpha (pSGV) vector, which contains the Venezuelan equine encephalitis virus nsP1-4 replication machinery genes and a 26S promoter that drives the expression of the GOI, are used as the saRNA expression vector. This vector and each GOI are digested with EcoRV and PacI restriction endonucleases to prepare the ends for cloning. Each digestion reaction is purified to eliminate the endonucleases and any unwanted DNA fragments. All 6 GOIs are ligated into the pSGV vector using ELECTROLIGASE® independently. The products are electroporated into TRANSFORMAX™ EPI300™ Electropcometent E. coli and resulting colonies screened for the presence of each GOI by isolating the plasmid DNA and analyzing via restriction fragment length polymorphism and next generation sequencing. Clones that have been ligated correctly with no mutations are made into glycerol stocks for long-term storage.

Amplification of IL-31 Replicon DNA. Samples taken from each glycerol stock for all 6 constructs will be struck for isolation on animal origin free LB agar plates, containing the kanamycin selection antibiotic, independently and allowed to replicate overnight. Isolated colonies will be lifted and used to inoculate animal origin free Terrific broth cultures containing the kanamycin selection antibiotic. The cultures will be incubated overnight at 37° C. with shaking to allow replication of the replicon plasmids containing the GOIs. The bacterial paste will be harvested by centrifugation and the NucleoBond Xtra Maxi EF kit will be used to isolate purified endotoxin-free plasmid DNA. This DNA will be tested for quantity by OD260, quality by measuring % supercoiled, and identity by Sanger or Lumina DNA Sequencing.

In vitro Transcription of IL-31 RNA. The purified DNA for each of the 6 constructs will be linearized by digesting the DNA with the NotI restriction endonuclease to aid in transcription. The linearized DNA will be transcribed into RNA overnight at 30° C. using the T7 DNA-dependent RNA polymerase and equal molar rNTPs. Subsequently, the transcription reaction will be treated with DNase I to digest the DNA template. The synthetic RNA product will be purified by filtration. The 5′ end of the uncapped RNA product will be enzymatically capped with guanyltransferase, rGTP, and SAM. The final capped synthetic RNA product will again be purified by filtration.

IL-31 SRV Formulation. The capped RNA transcripts of all 6 cIL-31 constructs will be encapsulated with Lipid Inorganic Nanoparticles (LION) and formulated according to the study protocol, TBD.

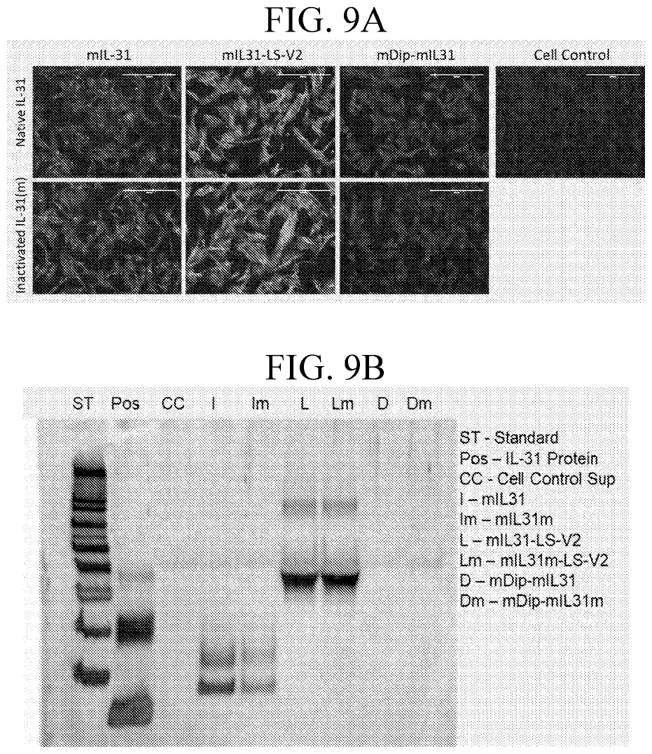

Expression of constructs. The six murine IL-31 Constructs described above were transfected into BHK-21 cells and protein expression was evaluated. Transfected cells were contacted with fluorescent anti-IL-31 antibodies. As shown by FIG. 9A and FIG. 9B below, IL-31 was expressed by the first four constructs in the supernatant, but no expression was detected in the supernatant for the two constructs encoding mutated diphtheria toxin or by the cell control.

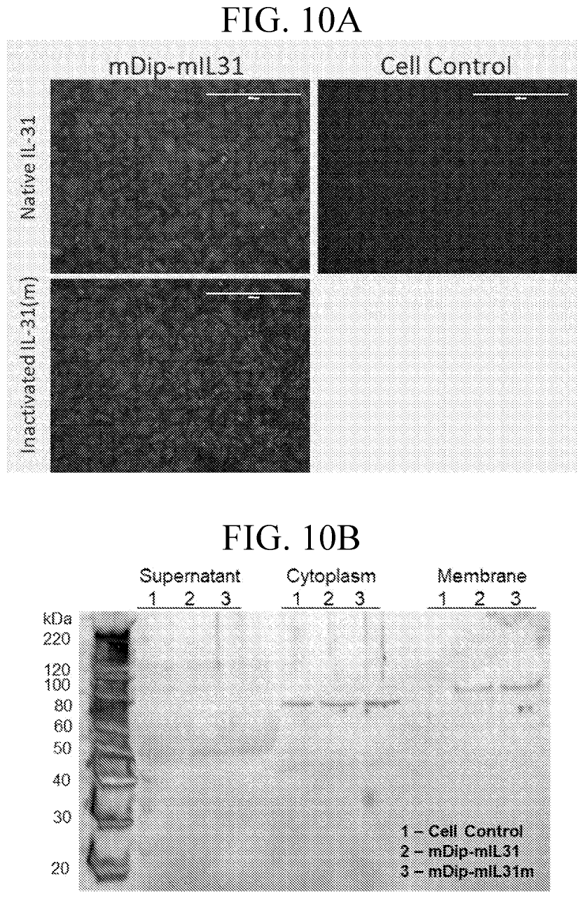

Transfected cells were also contacted with anti-diphtheria toxin (DT) antibodies. As shown by FIG. 10A and FIG. 10B.

These figures demonstrate that 4/6 constructs (wild-type, modified, and fused with LS) strongly expressed IL-31 in cell supernatant. In contrast, the 2/6 DT fusion constructs expressed antigens in the membranes.

In some embodiments, the saRNA as disclosed herein encodes an IL-31 product comprising residues that bind it to a host cell membrane and would prevent IL-31 interaction with IL31RA and subsequent recruitment of OSMR, thus preventing the expressed IL-31 for aggravating AD.

### Example 2. Construction of Canine IL-31 SRV

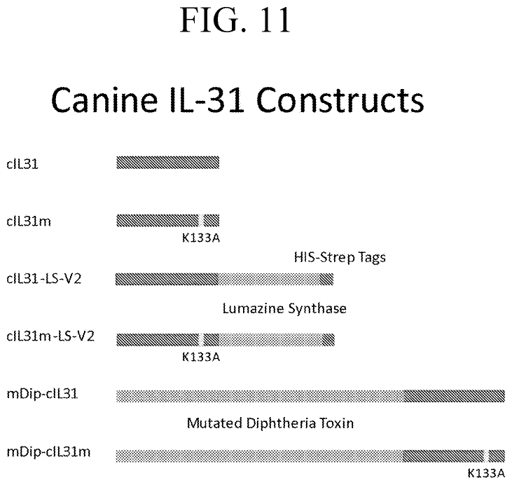

Design of 6 cIL-31 Genes of Interest. As shown by FIG. 11 below, two versions of the canine IL-31 gene of interest (GOI) are designed in silico, a native version (cIL-31) and an inactivated version with the K133A mutation (cIL-31m). These two IL-31 open reading frames (ORF) are also used to create 4 other GOIs. Two of these GOIs include fusing the 3′ ends of each IL-31 ORF (native and inactivated independently) to the 5′ end of the Lumazine Synthase ORF. These clones also contain 6×His and Strep tags on the 3′ end of the gene. The final two GOIs include fusing the 3′ end of the inactivated Diphtheria toxin ORF to the 5′ end of each IL-31 ORF (native and inactivated independently) with the IL-31 signal peptide removed; see FIG. 11.

The 5′ and 3′ ends of all 6 GOI are modified to contain additional nucleotides to aid in cloning into the replicon. On the 5′ end, the EcoRV restriction endonuclease site (GATATC) and Kozak sequence (GCCACC) are added upstream of the IL-31 or Diphtheria toxin start codon. On the 3′ end, the GCTGC sequence and PacI restriction endonuclease site (TTAATTAA) are added downstream of the IL-31 or Lumazine Synthase stop codon. All 6 GOIs are ordered as double stranded DNA fragments called gBlock™ from Integrated DNA Technologies.

Ligation of IL-31 Genes of Interest into VEE backbone. The pSGV_023_KAN Alpha (pSGV) vector, which contains the Venezuelan equine encephalitis virus nsP1-4 replication machinery genes and a 26S promoter that drives the expression of the GOI, are used as the replicon expression vector. This vector and each GOI are digested with EcoRV and PacI restriction endonucleases to prepare the ends for cloning. Each digestion reaction is purified to eliminate the endonucleases and any unwanted DNA fragments. All 6 GOIs are ligated into the pSGV vector using ELECTROLIGASE® independently. The products are electroporated into TRANSFORMAX™ EPI300™ Electropcometent E. coli and resulting colonies screened for the presence of each GOI by isolating the plasmid DNA and analyzing via restriction fragment length polymorphism and next generation sequencing. Clones that have been ligated correctly with no mutations are made into glycerol stocks for long-term storage.

Amplification of IL-31 Replicon DNA. Samples taken from each glycerol stock for all 6 constructs are struck for isolation on animal origin free LB agar plates, containing the kanamycin selection antibiotic, independently and allowed to replicate overnight. Isolated colonies are lifted and used to inoculate animal origin free Terrific broth cultures containing the kanamycin selection antibiotic. The cultures are incubated overnight at 37° C. with shaking to allow replication of the replicon plasmids containing the GOIs. The bacterial paste is harvested by centrifugation and the NucleoBond Xtra Maxi EF kit is used to isolate purified endotoxin-free plasmid DNA. This DNA is tested for quantity by OD260, quality by measuring % supercoiled, and identity by Sanger or Lumina DNA Sequencing.

In vitro Transcription of IL-31 RNA. The purified DNA for each of the 6 constructs is linearized by digesting the DNA with the NotI restriction endonuclease to aid in transcription. The linearized DNA is transcribed into RNA overnight at 30° C. using the T7 DNA-dependent RNA polymerase and equal molar rNTPs. Subsequently, the transcription reaction is treated with DNase I to digest the DNA template. The synthetic RNA product is purified by filtration. The 5′ end of the uncapped RNA product is enzymatically capped with guanyltransferase, rGTP, and SAM. The final capped synthetic RNA product is again purified by filtration.

IL-31 SRV Formulation. The capped RNA transcripts of all six cIL-31 constructs are encapsulated with Lipid Inorganic Nanoparticles (LION) and formulated according to the study protocol, TBD.

### Example 3. Evaluation of Three Candidate saRNA Vaccines in Mice

The inventors designed five different murine IL-31 constructs the inserts of which are described in FIG. 8. Subsequently, they evaluated the ability of saRNAs carrying these constructs to reduce pruritis for example by breaking tolerance to a murine self-protein mIL-31 without itself increasing pruritis. This work also permitted initial evaluation of the safety of these vaccines.

Three of the five vaccines were evaluated by administering the vaccines to groups of eight healthy breed, strain, age, and gender matched mice which were acclimated to study conditions for at least one day prior to start of this work.

Groups 1 and 2 received only PBS diluent and did not receive any vaccine.

In Groups 3, 4, and 5 mice were vaccinated twice with 5 ug/25 ul of the respective IL-31 constructs in 0.9% saline over a four week interval on Day 0 and Day 28.

A brief description of each of the five groups appears below.

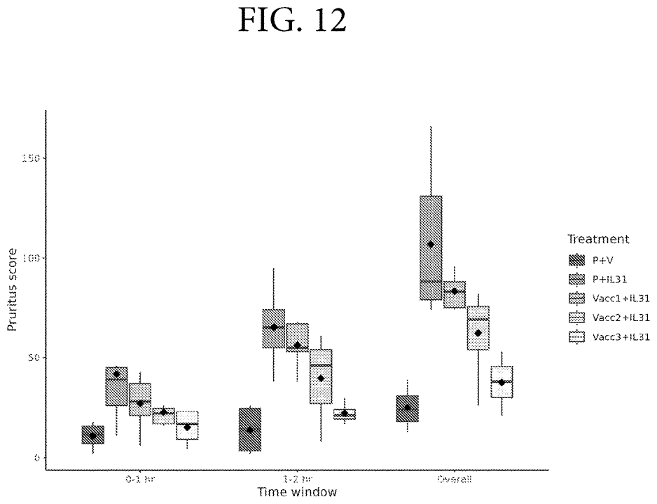

Group 1 (negative control). This group was injected with PBS only and was not challenged with IL-31. First bar in each set of five shown in FIG. 12.

Group 2 (challenge product control). This group was injected with PBS only, but unlike Group 1 was challenged with IL-31. Second bar in each set of five shown in FIG. 12.

Group 3. Vaccine 1. This group was injected with SRV carrying an insert encoding wild-type murine IL-31. Third bar in each set of five shown in FIG. 12.

Group 4. Vaccine 2. This group was injected with SRV carrying an insert encoding a protein comprising murine IL-31 fused to Lumazine synthase. Fourth bar in each set of five shown in FIG. 12.

Group 5. Vaccine 3. This group was injected with SRV carrying an insert encoding a protein comprising murine IL-31 fused to diphtheria toxin. Fifth bar in each set of five shown in FIG. 12.

IL-31 challenge. After vaccination, the animals in each of Groups 3 to 5 were challenged with IL-31 and pruritis scores were determined. Additionally, as a control, Group 2 mice, which were vaccinated with alphavirus vehicle only (no IL-31 coding sequences), were also challenged with IL-31. Group 1 mice were vaccinated with empty vehicle only and were not challenged with IL-31.

Pruritus scores were assessed at the time points after IL-3 challenge described in FIG. 12.

Results of vaccination on pruritis are shown in FIG. 12 and in the Table below.

| Test | Statistic | Degree of freedom | P-value |
|---|---|---|---|
| Kruskal-Wallis rank sum test | 18.89 | 4 | <0.001 |

Conover-Im an test

| Comparisons | P-value |
|---|---|
| P + IL31 − P + V | <0.001 |
| P + IL31 − Vacc1 + IL31 | 0.221 |
| P + V − Vacc1 + IL31 | <0.001 |
| P + IL31 − Vacc2 + IL31 | 0.003 |
| P + V − Vacc2 + IL31 | 0.015 |
| Vacc1 + IL31 − Vacc2 + IL31 | 0.026 |
| P + IL31 − Vacc3 + IL31 | <0.001 |
| P + V − Vacc3 + IL31 | 0.319 |
| Vacc1 + IL31 − Vacc3 + IL31 | 0.002 |
| Vacc2 + IL31 − Vacc3 + IL31 | 0.107 |

As apparent from FIG. 12 vaccination with Vaccine 1 (Vacc1), Vaccine 2 (Vacc2) and Vaccine 3 (Vacc3) reduced pruritis scores in vaccinated animals with Vaccine 3 providing the most significant reduction.

Comparison of the control (P+IL31) to each of these vaccines showed that the reductions in pruritis were significant for animals receiving either Vaccine 2 (P-value=0.003) and Vaccine 3 (P-value=<0.001).

### Other Protocol Details.

Animals: Female (4 weeks) old mice (C57BL16) were obtained from The Jacksons Laboratory (ME, USA). Mice were housed in groups of four on corn cob bedding and kept on a 12-hour light-dark cycle with lights off at 1800 hours. All mice had ad libitum access to chow (Purina LabDiet 5001) and water. Cage changes were performed weekly, and all animals received autoclaved cardboard enrichment for the duration of the study. Standard housing, in conformity with the regulation for animal welfare and the standard procedures at the experimental site, were provided. All animal experiments were approved by the Institutional Animal Care and Use Committees at North Carolina State University

Murine IL31 was purchased from R&D (Cat. #3028-ML Lot NJW1420041 and NJW1623011).

Safety assessment. Following the administration of the vaccine test products and control compositions, mice were observed any abnormal observations were recorded. These observations included body weight, behavioral change, respiratory signs, digestive signs, locomotion disorders, and skin or appendage disorders. Observations were made hourly for the first 6-8 hours after administration, then daily for the next two weeks, and then weekly until the end of the study. Local tolerance, including presence or degree of swelling, oedema, abscess, hair loss and pain at the injection site were assessed at time of injection or in the following days.

Behavior: Before vaccine and control injections, mice in an acrylic chamber were acclimated in a recording condition for at least 30 minutes. All animals were habituated to the handling and restraint necessary for the performance of all tests and injections—mice we randomly selected, and each cage of mice received an intramuscular injection of vaccine/placebo/mIL31. Spontaneous behaviors following injection of test vaccine or control into the tail vein (5 ug/2511 saline) were recorded by a video camera (GoPro and, Japan) for 2 hours to evaluate scratching behaviors. The number of scratching bouts was recorded throughout the body, neck, and cheek using hind paw directed behavior.

Statistical analysis of pruritis scores was performed using R software and the results appear in FIG. 12. Outlier data points that disproportionate affected the results for a group and hinder intergroup comparisons were omitted. Specifically, outliers were selected as follows: In the challenge group, P+IL31 (value excluded: 64): the excluded mouse does not seem sensitive to IL31 and has very weak scratching both during the first hour and during the second hour. In the Vacc1+IL31 group, values excluded: 128 and 125. In the Vacc2+IL31 group, Values excluded: 159 In the Vacc3+IL31 group, values excluded: 108, 132,139. In these last 3 groups, the criteria was: all mice with values greater than 100 were excluded. The level of significance was set at P<0.05. Boxplot presenting mean, median and interquartile range per time window and treatment group is shown in FIG. 12. To evaluate the treatment effect on the pruritus score, a Kruskal-Wallis rank sum test on data for the whole period and a Conover-Iman test provided pairwise comparisons for all treatment groups have been performed.

These results show that immunization with alphavirus vectors encoding immunogenic IL-31 constructs reduce the severity of pruritis.

### Example 4. Evaluation of Candidate Vaccines in Dogs with saRNA Expressing cIL-31 Challenge

The ability of several different saRNA vaccines to induce neutralizing antibodies to canine cytokine IL-31 and protect dogs against pruritus is evaluated. One challenge of this study was to determine whether a vaccine to cIL-31 could break immunological tolerance to a self-protein, cIL-31, without itself inducing pruritus. The primary objective of this study is to evaluate if candidate cIL-31 vaccines are efficient to induce anti-cIL-31 antibodies and to reduce the severity or prevent IL-31-induced pruritus. Secondary objectives are to evaluate the safety of the candidate vaccines and the duration of the protection against the pruritogen effect of cIL-31.

Eight groups of 6 healthy dogs, which are breed, strain, age, and gender matches, are used. Animals are acclimated for 1 or more days prior to start of the study. Standard housing, in conformity with the regulation for animal welfare and the standard procedures at the experimental site, is provided.

Groups 1 and 2 receive 1 ml PBS (alphavirus diluent) in lieu of vaccine three times with a 4-week interval (on Day 0, Day 28, and D56). Group 1 is a negative control group that receives no vaccine and that is not challenged with cIL-31. Group 2 is a challenge product control group that receives no vaccine and that is challenged with cIL-31.

Each of Groups 3 to 8 receive 300 μg in a 1 mL volume of the alphavirus constructs respectively shown in FIG. 11.

Group 3 receives 300 μg in a 1 mL volume of an alphavirus construct encoding wild-type cIL-31, see the first construct described by FIG. 11.

Group 4 receives an alphavirus construct encoding cIL-31m carrying the K133A mutation; see the second construct in FIG. 11.

Group 5 receives 300 μg in a 1 mL volume of an alphavirus construct encoding Lumazine Synthase fused with wild-type cIL-31, see the third construct described by FIG. 11.

Group 6 receives 300 μg in a 1 mL volume of an alphavirus construct encoding Lumazine Synthase fused with cIL-31m carrying the K133A mutation, see the fourth construct described by FIG. 11.

Group 7 receives 300 μg in a 1 mL volume of an alphavirus construct encoding Diphtheria toxoid fused with wild-type cIL-31, see the fifth construct described by FIG. 11.

Group 8 receives 300 μg in a 1 mL volume of an alphavirus construct encoding Diphtheria toxoid fused with cIL-31m carrying the K133A mutation, see the last construct described by FIG. 11.

IL-31 alphavirus vaccines are stored at <−50° C.

IL-31 challenge. Three weeks after the second injection of the alphavirus vaccine (Day 43), the dogs are challenged with an IV or SC administration of a recombinant cIL-31 in an amount of 2.5 μg/kg that induces a measurable episode of pruritus detectable by repeated scratching. The intensity and frequency of the pruritus is measured and compared between groups. The person in charge of evaluating the intensity of pruritus is not aware of the treatment received by the dogs.

Dogs in groups showing successful reduction of AD are again challenged with the same dosage of cIL-31 on Day 70 followed by assessment of the intensity and frequency of pruritus.

Canine serum is collected periodically including on Days 0 and 21 prior to administration of the cIL-31 vaccines, and pre- and post-cIL-31 challenge. Antibodies to cIL-31 are determined at each time point.

Safety assessment. Any abnormal observation is recorded hourly in the 8 hours following administrations of test products and then daily until the end of the study. The presence of degree of pruritus including the development of pruritic reaction administration is closely monitored in the hours following administration of test products, loss of appetite, behavioral change, respiratory signs, digestive signs, locomotion disorders, skin or appendage disorders, any other abnormal sign are monitored. Local tolerance at the site of injection is assessed including presence or degree of swelling, oedema, abscess, hair loss and pain at time of injection or in the following days.

### Example 5. Evaluation of Candidate saRNA Vaccines in Mice or Dogs with Dust Mite Allergen Challenge

Mice and dogs are tested and evaluated as disclosed in Examples 3 and 4 except that they are challenged with dust mite allergens instead of with mIL-31 or cIL-31. For mice, 2.5 μg/kg of dust mite allergen is administered to each mouse. The amount of allergen extracts used for each dog is 5 mg diluted in 0.4-0.45 mL of paraffin oil.

Accession numbers of IL-31, IL31RA, and Oncostatin M beta nucleic acids and amino acid sequences are provided below. The sequences of precursor, processed, and mature forms of the proteins named below as well as their variants; their modified or glycosylated residues of these proteins; and the subcellular locations of these proteins are described by and incorporated by reference to the database entries accessed by these accession numbers (last accessed Aug. 12, 2022). For example, UNIPROT primary accession number C7G0W1 (in section 2 below) describes a sequence of canine IL-31, its subcellular location, and PTM/Processing. External and cytoplasmic domains of such proteins are typically separated by the transmembrane domain. An SRV construct may comprise polynucleotide sequences encoding IL-31 or IL21RA proteins or their immunogenic segments or variants. Polynucleotides encoding the various domains of IL-31 or IL31RA, including signal or leader peptides or the domains or regions of an IL-31 or IL31RA protein described in by the accession numbers described below for SEQ ID NOS: 1-12 may be omitted or included in a construct.

Terminology. Terminology used herein is for the purpose of describing particular embodiments only and is not intended to be limiting of the invention.

The headings (such as “Background” and “Summary”) and sub-headings used herein are intended only for general organization of topics within the present invention and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by deletion of http: or by insertion of a space or underlined space before www. In some instances, the text available via the link on the “last accessed” date may be incorporated by reference.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology. As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

Sequence information. The section below describes accession numbers describing the nucleotide (e.g. RNA or DNA) or amino acid (protein) sequences of IL-31 and its receptors. However, the nucleotides and amino acid sequences disclosed herein are not limited to these sequences. In some instances, these sequences can be further modified to produce variant sequences, such as sequences having 1, 2, 3, 4, 5, 6, 7, 8, 9, 10 or more deletions, substitutions, or insertions of nucleotides or amino acid residues or sequences having a certain degree of sequence identity to the sequences described by these accession, numbers. Other modifications include glycosylation or other chemical of amino acid residues, removal of glycosylation from a residue or its replacement by a non-glycosylated amino acid residue, or structural modifications including multimerization or alternative protein folding.

Amino acid sequences may be deduced from the corresponding RNA or nucleic acid sequences using the genetic code; polynucleotide sequences that encode the amino acid sequences described below may be deduced from the amino acid sequences using the genetic code. All the accession numbers described below are incorporated by reference to the GENBANK database as last accessed Aug. 19, 2022. Information disclosed by the GENBANK accession number entries that is not reproduced below is expressly incorporated by reference.

1. SEQ ID NO: 1. Canine IL-31. NCBI Reference Sequence: NP_001159386.1.

| LOCUS | NP_001159386 159 aa linear MAM 20-FEB-2022 | |
|---|---|---|
| DEFINITION | interleukin-31 precursor [Canis lupus familiaris]. | |
| ACCESSION | NP_001159386 | |
| VERSION | NP_001159386.1 | |
| DBSOURCE | REFSEQ: accession NM_001165914.1 | |
| KEYWORDS | RefSeq. | |
| SOURCE | Canis lupus familiaris (dog) | |
| ORGANISM | Canis lupus familiaris | |
| FEATURES | Location/Qualifiers | |
| source | 1..159 | |
| | /organism=“Canis lupus familiaris” | |
| | /sub_species=“familiaris” | |
| | /db_xref=“taxon:9615” | |
| | /chromosome=“26” | |
| | /map=“26” | |
| Protein | 1..159 | |
| | /product=“interleukin-31 precursor” | |
| | /calculated_mol_wt=15374 | |
| sig_peptide | 1..23 | |
| | /inference=“COORDINATES: ab initio prediction:SignalP:4.0” | |
| | /calculated_mol_wt=2555 | |
| Region | 24..153 | |
| | /region_name=“IL31” | |
| | /note=“Interleukin 31; pfam15209” | |
| | /db_xref=“CDD:405816” | |
| CDS | 1..159 | |
| | /gene=“IL31” | |
| | /coded by=“NM_001165914.1:30..509” | |
| | /db_xref=“GeneID:100302725” | |
| | /db_xref=“VGNC:VGNC:41980” | |
| ORIGIN | | |
| | 1 | mlshtopsrf alflicsmet lisshmapth qlppsdvrki ilelqplsrg lledyqkket |
| | 61 | gypesnrtil lcltsdsqpp rinssailpy frairplsdk niidkiieql dklkfqhepe |
| | 121 | teisvpadtf ecksfiltil qqisaclesv fksinsgpq |
| // | | |

2. SEQ ID NO: 2. Interleukin 31 [Mus musculus]. GenBank: AAS82846.1.

| LOCUS | AAS82846 163 aa linear ROD 10-JUL-2004 | |
|---|---|---|
| DEFINITION | interleukin 31 [Mus musculus. | |
| ACCESSION | AAS82846 | |
| VERSION | AAS82846.1 | |
| DBSOURCE | accession AY509149.1 | |
| KEYWORDS | . | |
| SOURCE | Mus musculus (house mouse) | |
| ORGANISM | Mus musculus | |
| FEATURES | Location/Qualifiers | |
| source | 1..163 | |
| | /organism=“Mus musculus” | |
| | /strain=“BALB/c” | |
| | /db_xref=“taxon:10090” | |
| | /clone=“muzcytor17lig” | |
| | /tissue_type=“testis” | |
| Protein | 1..163 | |
| | /product=“interleukin 31” | |
| | /name=“four helical bundle cytokine” | |
| Region | 28..158 | |
| | /region name=“IL31” | |
| | /note=“Interleukin 31; pfam15209” | |
| | /db_xref=“CDD:405816” | |
| CDS | 1..163 | |
| | /gene=“I131” | |
| | /coded_by=“AY509149.1:53..544” | |
| ORIGIN | | |
| | 1 | mifhtgttkp tlvllccigt wlatcslsfg apiskedlrt tidllkqesq dlynnysikq |
| | 61 | asgmsadesi qlpcfsldre altnisviia hlekvkvlse ntvdtswvir wltniscfnp |
| | 121 | lnlnisvpgn tdesydckvf vltvlkqfsn cmaelqakdn ttc |

```
  3. SEQ ID NO: 3. Equus caballus. interleukin-31, partial [Equus caballus]; NCBI
        Reference Sequence: XP_023503836.1.
```

| LOCUS | XP_023503836 179 aa linear MAM 23-JAN-2 | |
|---|---|---|
| DEFINITION | interleukin-31, partial [Equus caballus]. | |
| ACCESSION | XP_023503836 | |
| VERSION | XP_023503836.1 | |
| DBLINK | BioProject: PRJNA19129 | |
| DESOURCE | REFSEQ: accession XM_023648068.1 | |
| KEYWORDS | RefSeq; includes ab initio. | |
| SOURCE | Equus caballus (horse) | |
| ORGANISM | Equus caballus | |
| FEATURES | Location/Qualifiers | |
| source | 1..179 | |
| | /organism=“Equus caballus” | |
| | /isolate=“Twilight” | |
| | /db_xref=“taxon:9796” | |
| | /chromosome=“8” | |
| | /sex=“female” | |
| | /breed=“thoroughbred” | |
| Protein | <1..179 | |
| | /product=“interleukin-31” | |
| Region | 50..175 | |
| | /region_name=“IL31” | |
| | /note=“Interleukin 31; pfam15209” | |
| | /db_xref=“CDD:405816* | |
| CDS | 1..179 | |
| | /gene=“IL31” | |
| | /coded by=“XM_023648068.1:<1..540” | |
| | /db_xref=“GeneID:102149194” | |
| | /db_xref=“VGNC:50942” | |
| ORIGIN | | |
| | 1 | atartrapgr gigsrrlwpq rrrrsrkprl pgstrfalfl lcclgtlmfs htgpiyqlqp |
| | 61 | keiqaiivel qnlskklldd ylnkekgvqk fdsdlpscft sdsqapgnin ssailpyfka |
| | 121 | ispslnndks lyiieqldkl nfqnapetsv smptdnferk rfiltilrwf snclehraq |
| // | | |

```
  4. SEQ ID NO: 4. PREDICTED: Felis catus interleukin 31 (IL31), mRNA; NCBI
        Reference Sequence: XM_045042055.1.
```

| | |
|---|---|
| LOCUS | XM_045042055 642 bp mRNA linear MAM 09-NOV-2021 |
| DEFINITION | PREDICTED: Felis catus interleukin 31 (IL31), mRNA. |
| ACCESSION | XM_045042055 |
| VERSION | XM_045042055.1 |
| DELINK | BioProject: PRJNA773801 |
| KEYWORDS | RefSeq; includes ab initio. |
| SOURCE | Felis catus (domestic cat) |
| ORGANISM | Felis catus |
| | - |
| FEATURES | Location/Qualifiers |
| source | 1..642 |
| | /organism=“Felis catus” |
| | /mol_type=“mRNA” |
| | /isolate=“Fca126” |
| | /db_xref=“taxon:9685” |
| | /chromosome=“D3” |
| | /sex=“female” |
| | /cell_type=“Fibroblast” |
| | /tissue_type=“Fibroblasts” |
| | /dev_stage=“adult” |
| gene | 1..642 |
| | /gene=“IL31” |
| | /note=“Derived by automated computational analysis using |
| | gene prediction method: Gnomon. Supporting evidence |
| | includes similarity to: 2 Proteins, and 74% coverage of |
| | the annotated genomic feature by RNAseq alignments” |
| | /db_xref=“GeneID:105261056” |
| | /db_xref=“VGNC:VGNC:80444” |
| CDS | 1..642 |
| | /gene=“IL31” |
| | /codon_start=1 |
| | /product=“interleukin-31” |
| | /protein_id=“XP_044897990.1” |
| | /db_xref=“GeneID:105261056” |
| | /db_xref=“VGNC:VGNC:80444” |
| | /translation=“MFKAKQNGDEAFDSPYSLWLSGPSPHTGMGILQKFPKWLQLGWA |
| | GGVWPRGPQHHFLSPGPARFALFLLCCMEILLPSHMAPTHRLQPSDVRKIILELRPMS |
| | KGLLQDYLKKEIGLPESNHSSLPCLSSDSQLPHINGSAILPYFRAIRPLSDKNTIDKI |
| | IEQLDKLKFQREPEAEVSMPADNFERKNFILAVLQQFSACLEHVLQSINSGPQ” |

```
  5. SEQ ID NO: 5. PREDICTED: Canis lupus familiaris interleukin 31 receptor A
      (IL31RA), transcript variant X1, mRNA; NCBI Reference Sequence: XM_038658911.1
```

| | |
|---|---|
| LOCUS | XM_038658911 8571 bp mRNA linear MAM 07-JAN-2021 |
| DEFINITION | PREDICTED: Canis lupus familiaris interleukin 31 receptor A |
| | (IL31RA), transcript variant X1, mRNA. |
| ACCESSION | XM_038658911 |
| VERSION | XM_038658911.1 |
| DBLINK | BioProject: PRJNA681562 |
| KEYWORDS | RefSeq. |
| SOURCE | Canis lupus familiaris (dog) |
| ORGANISM | Canis lupus familiaria |
| FEATURES | Location/Qualifiers |
| source | 1..8571 |
| | /organism=“Canis lupus familiaris” |
| | /mol_type=“mRNA” |
| | /isolate=“SID07034” |
| | /sub_species=“familiaris” |
| | /db_xref=“taxon:9615” |
| | /chromosome=“2” |
| | /sex=“male” |
| | /tissue_type=“fibroblast, soft palate” |
| | /dev_stage=“adult” |
| | /breed=“Labrador retriever” |
| gene | 1..8571 |
| | /gene=“IL31RA” |
| | /note=“Derived by automated computational analysis using |
| | gene prediction method: Gnomon. Supporting evidence |
| | includes similarity to: 100% coverage of the annotated |
| | genomic feature by RNAseg alignments, including 2 samples |
| | with support for all annotated introns” |
| | /db_xref=“GeneID:487212” |
| | /db_xref=“VGNC:41981” |
| CDS | 301..2664 |
| | /gene=“IL31RA” |
| | /codon_start=1 |
| | /product=“interleukin-31 receptor subunit alpha isoform |
| | X1” |
| | /protein id=“XP_038514839.1” |
| | /db xref=“GeneID:487212” |
| | /db xref=“VGNC:VGNC:41981” |
| | /translation=“MMWAKVLWMLLLLCKLSLAVLPAKPENISCIFYYEENFTCTWSP |
| | EKEASYTWYKVKRTYSYGYKSDICSTDNSTRGNHASCSFLPPTITNPDNYTIQVEAQN |
| | ADGIMKSDITYWNLDAIMKIEPPEIFSVKSVLGIKRMLQIKWIRPVLAPHSSTLKYTL |
| | RFRTINSAYWMEVNFTKEDIDRDETYNLTELQAFTEYVMTLRCAPAESMFWSGWSQEK |
| | VGTTEEEAPYGLDLWRVLKPAMVDGRRPVQLMWKKATGAPVLEKALGYNIWYFPENNT |
| | NLTETVNTTNQTHELYLGGKTYWVYVVSYNSLGESPVATLRIPALNEKTFQCIEAMQA |
| | CLTQDQLVVEWQSSAPEVDTWMVEWFPDVDSEPSSFSWESVSQARNWTIQKDELKPLW |
| | CYNISVYPVLRDRVGQPYSTQAYVQEGNGYCQKTVFPNKVIHRVPTLPLSSTSGTGFA |
| | LSTSHNLCGERIYSAVIQCLLVFSLSCKVPSAGPVTQADSIGVKTVTITWKEIPKSKR |
| | NGFIKNYTIFYQAEDGKEFSKTVNSNILQYRLESLTRRTSYSLQVMASTNAGGTNGTK |
| | INFKTLSISVLEIFFITSLVGGGFLILIMLTVAYGLKKPNKLKHLCWPDVPNPAESSI |
| | ATWRGDDFKDKLNLKESDDPVNMEEDQVLKPYSAPTDFIDKLVVNFENFLEEVSTEEL |
| | GKSQENILKEEKNKHVTSPYCLHHPPISTEIPQRKPQQLCSRIPEGTCSETKEQLFSS |
| | VQSLGPDHLCEEGEPNPYLKNSVTTREFVCLKNFKTKPREKESVAVA” |

```
  6. SEQ ID NO: 6. Mus musculus interleukin 31 receptor A (I131ra), mRNA; NCBI
        Reference Sequence: NM_139299.2
```

| | |
|---|---|
| LOCUS | NM_139299 3680 bp mRNA linear ROD 22-APR-2022 |
| DEFINITION | Mus musculus interleukin 31 receptor A (I131ra), MRNA. |
| ACCESSION | NM_139299 |
| VERSION | NM_139299.2 |
| KEYWORDS | RefSeq; RefSeq Select. |
| SOURCE | Mus musculus (house mouse) |
| ORGANISM | Mus musculus |
| FEATURES | Location/Qualifiers |
| source | 1..3680 |
| | /organism=“Mus musculus” |
| | /mol_type=“mRNA> |
| | /strain=“C57BL/6” |
| | /db_xref=“taxon:10090” |
| | /chromosome=“13” |
| | /map=“13” |
| gene | 1..3680 |
| | /gene=“I131ra” |
| | /gene_synonym=“CRL3; GLM-R; Glmx; GPL; NR10” |
| | /note=“interleukin 31 receptor A” |
| | /db_xref=“GeneID:218624” |
| | /db_xref=“MGI:MGI:2180511” |
| exon | 1..25 |
| | /gene=“I131ra” |
| | /gene_synonym=“CRL3; GLM-R; Glmr; GPL; NR10” |
| | /inference=“alignment:Splign:2.1.0″ |
| exon | 26..189 |
| | /gene=“Il31ra” |
| | /gene_synonym=“CRL3; GLM-R; GImr; GPL; NR10” |
| | /inference=“alignment:Splign:2.1.0” |
| exon | 190..397 |
| | /gene=“I131ra” |
| | /gene_synonym=“CRL3; GLM-R; Glmr; GPL; NR10” |
| | /inference=“alignment:Splign:2.1.0” |
| misc_feature | 392..394 |
| | /gene=“I131ra” |
| | /gene_synonym=“CRL3; GLM-R; Glmr; GPL; NR10” |
| | /note=“upstream in-frame stop codon” |
| exon | 398..476 |
| | /gene=“I131ra” |
| | /gene_synonym=“CRL3; GLM-R; Glmr; GPL; NR10” |
| | /inference=“alignment:Splign:2.1.0” |
| CDS | 422..2572 |
| | /gene=“I131ra” |
| | /gene_synonym=“CRL3; GLM-R; Glmr; GPL; NR10” |
| | /note=“gp130-like monocyte receptor; interleukin-31 |
| | receptor subunit alpha: interleukin 31RA; class I cytokine |
| | receptor; mGLM-R; IL-31RA; zcytoR17; IL-31R-alpha; |
| | gp130-like receptor; IL-31R subunit alpha; novel cytokine |
| | receptor 10; IL-31 receptor subunit alpha; cytokine |
| | receptor NR10; interleukin-31 receptor subunit alpha short |
| | peptide” |
| | /codon start=1 |
| | /product=“interleukin-31 receptor subunit alpha precursor” |
| | /protein id=“NP_647460.2” |
| | /db_xref=“CCDS:CCDS36781.1” |
| | /db_xref=“GeneID:218624” |
| | /db_xref=“MGI:MGI:2180511” |
| | /translation=“MWTLALWAFSFLCKESLAVLPTKPENISCVFYFDRNLTCTWRPE |
| | KETNDTSYIVILTYSYGKSNYSDNATEASYSFPRSCAMPPDICSVEVQAQNGDGKVKS |
| | DITYWHLISIAKTEPPIILSVNPICNRMFQIQWKPREKTRGFPLVCMLRFRTVNSSHW |
| | TEVNFENCKQVCNLTGLQAFTEYVLALRFRFNDSRYWSKWSKEETRVTMEEVPHVLDL |
| | WRILEPADMNGDRKVRLLWKKARGAPVLEKTFGYHIQYFAENSTNLTEINNITTQQYE |
| | LLLMSQAHSVSVTSFNSLGKSQEAILRIPDVHEKTFQYIKSMKAYIAEPLLVVNWQSS |
| | IPAVDTWIVEWLPEAAMSKFPALSWESVSQVTNWTIEQDKLKPFTCYNISVYPVLGHR |
| | VGEPYSIQAYAKEGTPLKGPETRVENIGLRTATITWKEIPKSARNGFINNYTVFYQAE |
| | GGKELSKTVNSHALOCDLESLTRRTSYTVWVMASTRAGGINGVRINFKTLSISVFEIV |
| | LLTSLVGGGLLLLSIKTVTFGLRKPNRLTPLCCPDVPNPAESSLATWLGDGFKKSNMK |
| | ETGNSGDTEDVVLKPCPVPADLIDKLVVNFENFLEVVLTEEAGKGQASILGGEANEYV |
| | TSPSRPDGPPGESFKEPSVLTEVASEDSHSTCSRMADEAYSELARQPSSSCQSPGLSP |
| | PREDQAQNPYLKNSVTTREFLVHENIPEHSKGEV” |
| sig_peptide | 422..475 |
| | /gene=“Il31ra” |
| | /gene_synonym=“CRL3; GLM-R; Glmr; GPL; NR10” |
| | /inference=“COORDINATES: ab initio prediction:SignalP:4.0” |
| misc_feature | 527..529 |
| | /gene=“I131ra” |
| | /gene_synonym=“CRL3; GLM-R; Glmr; GPL; NR10” |
| | /note=“N-linked (GlcNAc...) asparagine. |
| | /evidence=ECO:0000255; propagated from |
| | UniProtKB/Swiss-Prot (Q8K5B1.3); glycosylation site” |
| misc_feature | 563-565 |
| | /gene=“Il31ra″ |
| | /gene_synonym=“CRL3; GLM-R; Glmr; GPL; NR10” |
| | /note=“N-linked (GlcNAc...) asparagine. |
| | /evidence=ECO:0000255; propagated from |
| | UniProtKB/Swiss-Prot (Q8K5B1.3); glycosylation site” |
| misc_feature | 611..613 |
| | /gene=“I131ra” |
| | /gene_synonym=“CRL3; GLM-R; Glmr; GPL; NR10” |
| | /note=“N-linked (GlNAc...) asparagine. |
| | /evidence=ECO:0000255; propagated from |
| | UniProtKB/Swiss-Prot (Q8K5B1.3); glycosylation site″ |
| misc_feature | 1565..1567 |
| | /gene=“I131ra” |
| | /gene_synonym=“CRL3; GLM-R; Glmr; GPL; NR10” |
| | /note=“N-linked (GlcNAc...) asparagine. |
| | /evidence=ECO:0000255; propagated from |
| | UniProtKB/Swiss-Prot (Q8K5B1.3); glycosylation site” |
| misc_feature | 1919..1981 |
| | /gene=“I131ra” |
| | /gene synonym=“CRL3; GLM-R; Glmr; GPL; NR10” |
| | /note=“propagated from UniProtKB/Swiss-Prot (Q8K581.3); |
| | transmembrane region” |
| misc_feature | 2285..2344 |
| | /gene=“I131ra” |
| | /gene_synonym=“CRL3; GIM-R; Glmr; GPL; NR10” |
| | /note=“propagated from UniProtKB/Swiss-Prot (Q8K5B1.3); |
| | Region: Disordered. /evidence=ECO:0000256\|SAM:MobiDB-lite” |
| misc_feature | 2363..2509 |
| | /gene=“Il31ra” |
| | /gene_synonym=″CRL3; GLM-R; Glmr; GPL; NR10″ |
| | /note=“propagated from UniProtKB/Swiss-Prot (Q8KSB1.3); |
| | Region: Disordered. /evidence=ECO:0000256\|SAM:MobiDB-lite″ |
| exon | 477..594 |
| | /gene=“I131ra” |
| | /gene_synonym=“CRL3; GLM-R; Glmr; GPL; NR10” |
| | /inference=“alignment:Splign:2.1.0” |
| exon | 595..758 |
| | /gene=“I131ra” |
| | /gene_synonym=“CRL3; GLM-R; Glmr; GPL; NR10” |
| | /inference=“alignment:Splign:2.1.0” |
| exon | 759..901 |
| | /gene=“Il31ra” |
| | /gene_synonym=“CRL3; GLM-R; Glmr; GPL; NR10” |
| | /inference=″alignment:Splign:2.1.0” |
| exon | 902..1055 |
| | /gene=“I131ra” |
| | /gene_synonym=“CRL3; GLM-R; Glmr; GPL; NR10” |
| | /inference=“alignment:Splign:2.1.0” |
| exon | 1056..1135 |
| | /gene=“Il31ra” |
| | /gene_synonym=“CRL3: GLM-R; Glmr; GPL; NR10” |
| | /inference=“alignment:Splign:2.1.0” |
| exon | 1136..1352 |
| | /gene=“I131ra” |
| | /gene_synonym=“CRL3; GLM-R; Glmr; GPL; NR10” |
| | /inference=“alignment:Splign:2.1.0” |
| exon | 1641..1787 |
| | /gene=“I131ra” |
| | /gene_synonym=“CRL3; GLM-R; Glmr; GPL; NR10” |
| | /inference=“alignment:Splign:2.1.0” |
| exon | 1788..1928 |
| | /gene=“I131ra” |
| | /gene_synonym=“CRL3; GLM-R; Glmr: GPL; NR10” |
| | /inference=“alignment:Splign:2.1.0” |
| exon | 1929..2022 |
| | /gene=“I131ra” |
| | /gene_synonym=“CRL3; GLM-R; Glmr; GPL; NR10” |
| | /inference=“alignment:Splign:2.1.0” |
| exon | 2023..2104 |
| | /gene=“I131ra” |
| | /gene_synonym=“CRL3; GLM-R; Glmr; GPL; NR10” |
| | /inference=“alignment:Splign:2.1.0” |
| exon | 2105..3680 |
| | /gene=“I131ra” |
| | /gene_synonym=“CRL3; GLM-R; Glmr; GPL; NR10” |
| | /inference=“alignment:Splign:2.1.0” |

```
  7. SEQ ID NO: 7. PREDICTED: Equus caballus interleukin 31 receptor A (IL31RA),
          mRNA; NCBI Reference Sequence: XM_023625297.1
```

| LOCUS | XM_023625297 7811 bp mRNA linear MAM 23-JAN-2018 | |
|---|---|---|
| DEFINITION | PREDICTED: Equus caballus interleukin 31 receptor A (IL31RA), mRNA. | |
| ACCESSION | XM_023625297 | |
| VERSION | XM_023625297.1 | |
| DBLINK | BioProject: PRJNA19129 | |
| KEYWORDS | RefSeq; corrected model; includes ab initio. | |
| SOURCE | Equus caballus (horse) | |
| ORGANISM | Equus caballus | |
| | --------- ----------- ------ | |
| FEATURES | Location/Qualifiers | |
| source | 1.7811 | |
| | /organism=“Equus caballus” | |
| | /mol_type=“mRNA” | |
| | /isolate=“Twilight” | |
| | /db_xref=“taxon:9796” | |
| | /chromosome=“21” | |
| | /sex=“female” | |
| | /breed=“thoroughbred” | |
| gene | 1..7811 | |
| | /gene=“IL31RA” | |
| | /note=“Derived by automated computational analysis using | |
| | gene prediction method: Gnomon. Supporting evidence | |
| | includes similarity to: 8 Proteins, and 98% coverage of | |
| | the annotated genomic feature by RNAseq alignments” | |
| | /db_xref=“GeneID:100062017” | |
| | /db_xref=“VGNC:VGNC:19028” | |
| CDS | 1..2238 | |
| | /gene=“IL31RA” | |
| | /note=“The sequence of the model RefSeq protein was | |
| | modified relative to its source genomic sequence to | |
| | represent the inferred CDS: substituted 2 bases at 2 | |
| | genomic stop codons” | |
| | /codon_start=1 | |
| | /transl_except=(pos:31..33,aa:OTHER) | |
| | /transl_except=(pos:40..42,aa:OTHER) | |
| | /product=“LOW QUALITY PROTEIN: interleukin-31 receptor | |
| | subunit alpha” | |
| | | /protein id=″XP_023481065.1″ |
| | | /db_xref=″GeneID:100062017″ |
| | | /db_xref=″VGNC:VGNC:19028″ |
| | | /translation=″MAIQLSHQPAXDSXKTMWAWTLWMLPLICKFSLAVLLAKPENIS |
| | | CIFYYERNFTCTWSPEKEASDTWYTVKRTYCFGRKSDHCTTGNSTSTARTSCSFYPPP |
| | | ITIPDNYTIQVEAQNADGIIKSDITQWALDTIVKTEPPEIYSVKPVLGIKRMVQIKWK |
| | | RPVLAPKSTTLKYRLAFRTVNSAGWMEVNFKEGSNGDTTYNLTGLQAFTEYVVALQCV |
| | | AEESVFWSGWSQEKMGTTEEEAPEGLDLWRVLRPAMVDGRRPVQLLWKKARGAPVLEK |
| | | TLGYNIQYFPENNINLTKTVNTTNQQLDLYLGGETYWVSVISYNSLGESSLATLRIPA |
| | | IDEKPFQCIEAMHSCLTQDQLVVEWQSSASEVDTWMVEWFPVLDSEPSIFSWESVSGA |
| | | RNWTIQQDELAPFRCYNISVYPMLQDQVGEPYSIQAYVKEGIPSVGPVSKVENIGVKT |
| | | VTITWKEIPTERRNGFISNYTIFYQAEDGKEFSKIVNSSILQYGLESLTANTSYTVQV |
| | | MASTSAGGINGTRINFKTLSISVFEIFLITSLVGGSLLILIIPTVAYGLKKPNKLKHL |
| | | CWPDVPNPAESSIAMWRGDNFEDKLNVKEFDDSVNMEEERILKPCSVPSDLIDKLVVD |
| | | FENFLEEFSTEEPGKGQENILGGEKNEYVTSPYRPYCPTGKHFKELPLATEIPSRKSQ |
| | | YLSSGVPEGTSSEDKEQLLSSASSLGLDHLCEEGAPNPYLKNSVTTREFLVPEKLPDQ |
| | | TKREV” |

```
  8. SEQ ID NO: 8. PREDICTED:Felis catus interleukin 31 receptor A (IL3IRA), transcript
        variant X1, mRNA; NCBI Reference Sequence: XM_019834303.3
```

| | |
|---|---|
| LOCUS | XM_019834303 5818 bp MRNA linear MAM 09-NOV-2021 |
| DEFINITION | PREDICTED: Felis catus interleukin 31 receptor A (IL31RA) , |
| | transcript variant X1, mRNA. |
| ACCESSION | XM_019834303 |
| VERSION | XM_019834303.3 |
| DBLINK | BioProject: PRJNA773801 |
| KEYWORDS | Refseq. |
| SOURCE | Felis catus (domestic cat) |
| ORGANISM | Felis catus |
| FEATURES | Location/Qualifiers |
| source | 1..5818 |
| | /organism=“Felis catus” |
| | /mol_type=“mRNA” |
| | /isolate=“Fca126” |
| | /db_xref=“taxon:9685” |
| | /chromosome=“A1” |
| | /sex=“female” |
| | /cell_type=“Fibroblast” |
| | /tissue_type=“Fibroblast” |
| | /dev_stage=“adult” |
| gene | 1..5818 |
| | /gene=“IL31RA” |
| | /note=“Derived by automated computational analysis using |
| | gene prediction method: Gnomon. Supporting evidence |
| | includes similarity to: 4 Proteins, and 100% coverage of |
| | the annotated genomic feature by RNAseq alignments, |
| | including 14 samples with support for all annotated |
| | introns” |
| | /db_xref=“GeneID:101095878” |
| | /db_xref=“VGNC:VGNC:62911” |
| gene | 1255..3426 |
| | /gene=“IL31RA” |
| | /codon_start=1 |
| | /product=“interleukin-31 receptor subunit alpha isoform |
| | X1” |
| | - - - |
| | /protein id=“XP_019689862.3” |
| | /db_xref=“GeneID:101095878” |
| | /db_xref=“VGNC:VGNC:62911” |
| | /translation=“MMWAHALWTLLLLCKFSLAVLPAKPENISCVFYYEENFTCTWSP |
| | EKEASYTWYKVKRTYSYGYKSDICPSDNSTRGNHTFCSFLPPTITNPDNYTIQVEAQN |
| | ADGIIKSDITHWSLDAITKIEPPEIFSVKPVLGVKRMVQIKWIRPVLAPVSSTLKYTL |
| | RFKTVNSAYWMEVNFTKEDIDRDETYNLTGLQAFTEYVLALRCATKESMFWSGWSQEK |
| | MGTTEEEAPHGLDLWRVLRPATVDGRRLVQLMWKKASGAPVLEKALGYNIWYFPENST |
| | NLTKTLNTTNEKLELYLGGKTYWVCVVSYNSLGESPVATLRIPAIDEKSFQCIEAMEA |
| | CLTQDQLVVEWRSSAPEVDTWMVEWFPDLDSEPSTFSWESVSQATNWTIKQDELKPFW |
| | CYNISVYPVLQDRVGKPFSIQAYVREGIPSAGPVTQVDNIGVKTVTITWKEIPKSQRN |
| | GFITNYTIFYQAEDGKEFSKTVNSNILQYDLESLTRKTSYSLQVMASTSAGGINGTTM |
| | NFKTLSISILEIFLIISLVGGGLLILIILSVAYGLKKPNRLKHLCWPDVPNPAESSIA |
| | TWRGDDFKDKINLKESDDPVNMEEDRVLKPYSSPRDLIDKLVVNFENFLEDVSTEELG |
| | KGQENILREFKNEYVTSPYRPYCPPISTEIPQRKSQQLCSRIPEGICLETTEQLLSSV |
| | PNLGPDRICEEGEPNPYLKNSVTTREFLTSEKLPEQTKREV” |
| polyA_site | 5818 |
| | /gene=“IL31RA″ |
| | /experiment=″COORDINATES: polyA evidence [ECO:0006239]” |
| ----- | |

```
  9. SEQ ID NO: 9. PREDICTED: Canis lupus familiaris oncostatin M receptor (OSMR),
        transcript variant X5, mRNA; NCBI Reference Sequence: XM_038663878.1
```

| | |
|---|---|
| LOCUS | XM_038663878 4278 bp mRNA liner MAM 07-JAN-2021 |
| DEFINITION | PREDICTED: Canis lupus familiaris oncostatin M receptor (OSMR) |
| | transcript variant X5, mRNA. |
| ACCESSION | KM_038663878 |
| VERSION | XM_038663878.1 |
| DBLINK | BioProject: PRJNA681562 |
| KEYWORDS | RefSeq. |
| SOURCE | Canis lupus familiaris (dog) |
| ORGANISM | Canis lupus familiaris |
| FEATURES | Location/Qualifiers |
| source | 1..4278 |
| | /organiam=“Canis lupus familiaris” |
| | /mol_type=“mRNA” |
| | /isolate=“SID07034” |
| | /sub_species=“familiaris” |
| | /db_xref=“taxon:9615” |
| | /chromosome=“4” |
| | /sex=“male” |
| | /tissue_type=“fibroblast, soft palate” |
| | /dev_stage=“adult” |
| | /breed=“Labrador retriever” |
| gene | 1..4278 |
| | /gene=“OSMR” |
| | /note=“Derived by automated computational analysis using |
| | gene prediction method: Gnomon. Supporting evidence |
| | includes similarity to: 3 ESTs, and 100% coverage of the |
| | annotated genomic feature by RNAseq alignments, including |
| | 19 samples with support for all annotated introns” |
| | /db_xref=“GeneID:489223” |
| | /db_xref=“VGNC:VGNC:44167” |
| CDS | 289..2640 |
| | /gene=“OSMR” |
| | /codon_start=1 |
| | /product=“oncostatin-M-specific receptor subunit beta |
| | isoform X4” |
| | /protein id=XP_038519806.1 |
| | /db_xref=″GeneID:489223* |
| | /db_xref=″VGNC:VGNC:44167 |
| | /translation= MQGQQLDPNVSMFNLHNVAFIRETGTNIYCKVDRGDDIKGIVLF |
| | VSKILEEPKDFSCETRDFQTLSCTWDPGRDTGLLKQLPQSYTLFESFSGKKTLCKHKG |
| | WCNWQVAPESQEMYNFTLTAENYLRKRSVHILFNLTHRVHPMAPFNVLFKDVSVTNAT |
| | MTWKVHSTGNYYTLLCQVELYGEGKVIQKHNVSVKVNGELVLSGLEPDTEYSAQVRCA |
| | NANHFWKWSEWTRQNFTTVEAAPSEAPDVWRNVKSVQGYCVVTLFWKPLSRLQANGEI |
| | LFYNIVVEDLDRPSGLQLLSVPAPANRTELTLDQRAYQIHVTANNSVGTSPASVLVVS |
| | GDPGDEEVEEERVKGTEDGFSLSWKPQPGNITGYIVEWCDRPRDPLCDLQWKHLGPNT |
| | TSTVISSDAFRPGVRYNFRIYGISTEMVPYLLEKKTGYSQELAPSDNPRVVMSNLTSH |
| | SFTLSWKDYSTESQPSFIRGYHVYLKSKAGQCHPGSEKAVLSDDSVCCKYKIDDPKQK |
| | MFVVGNLQPESFYEFLVTPYSAVGEGPHGAFTKVTTPDEYSPMLIRIILPMIFCILLI |
| | MILCYLKSQWMKEKCYPDIPDPYKSSVLSLIKSKENPRLTIMNIKDCVPDTIEVVNKH |
| | EGTGKSPTEPEAAKPTYLYLLPAEESYSGPGPCICFENFTYNQAASDSASCGHVPVPT |
| | KAPSQLGLLTSSENLLTALEKNCMNSLEEIPAGESSLNYVSQVASPVSGDKSSLPTNP |
| | PQPELCSEYKMQMAIPLGLAAPSSSGSSSLSSITLLDEGEHYH″ |

10. SEQ ID NO: 10. Mus musculus mRNA for oncostatin M receptor bets, complete cds;
GenBank: AB015978.1

| | |
|---|---|
| LOCUS | AB015978 4026 bp mRNA linear ROD 06-MAR-1999 |
| DEFINITION | Mus musculus mRNA for oncostatin M receptor beta, complete cds. |
| ACCESSION | AB015978 |
| VERSION | AB015978.1 |
| KEYWORDS | oncostatin M receptor beta; Osmr. |
| SOURCE | Mus musculus (house mouse) |
| ORGANISM | Mus musculus |
| FEATURES | Location/Qualifiers |
| source | 1..4026 |
| | /organism=“Mus musculus” |
| | /mol_type=“mRNA” |
| | /db_xref=“taxon:10090” |
| gene | 1..4026 |
| | /gene=“Osmr” |
| CDS | 780..3692 |
| | /gene=“Osmr” |
| | /codon_start=1 |
| | /product=“oncostatin M receptor beta” |
| | /protein_id=“BAA33725.1” |
| | /translation=″MAFSVVLHPAFLLAVLSLRASRSEVLEEPLPLTPEIHKVSFQLK |
| | LQEVNLEWTVPALTHEELNMIFQIEISRLNISNTIWVENYSTTYKREEAVRWNWTSDI |
| | PLECVKHFIRIRALVDDTKSLPQSSWGNWSSWKEVNAKVSVEPDKSLIFPKDKVLEEG |
| | SNVTICLMYGQNVYNVSCKLQDEPIHGEQLDSHVSLLKLNNVVFLSDTGTNINCQATK |
| | GPKRIFGTVLFVSKVLEEPKNYSCETRDFKTLDCSWEPGVDTTLTWRKQRFQNYTLCE |
| | SFSKRCEVSNYRNSYTWQITEGSQEMYNFTLTAENQLRKRSVNINFNLTHRVHPKAPQ |
| | DVTLKIIGATKANMTWKVHSHGNNYTLLCQVKLQYGEVIHEHNVSVHMSANYLFSDLD |
| | PDTKYKAFVRCASANHFWKWSDWTQKEFSTPETAPSQALDVWRQVWSENGRRIVTLFW |
| | KPLLKSQANGKIISYNIVVENEAKPTESEHYCVWAPALSTNLSLDLQPYKIRITANNS |
| | MGASPESLMVLSNDSGHEVKEKTIKGIKDAFNISWEPVSGDTMGYVVDWCAHSQDQRC |
| | DLQWKNLGPNTTSTTITSDDFKPGVRYNFRIFERSVEHEARLVEKQRGYTQELAPLVN |
| | PKVEIPYSTPNSFVLRWPDYDSDFQAGFIKGYLVYVKSKEMQCNQPWERTLLPDNSVL |
| | CKYDINGSETKTLTVENLQPESLYEFFVTPYTSAGPGPNETFTKVTTPDARSHMLLQI |
| | ILPMTLCVLLSIIVCYWKSQWVKEKCYPDIPNPYKSSILSLIKSKKNPHLIMNVKDCI |
| | PDVLEVINKAEGSKTQCVGSGKLHIEDVPTKPPIVPTERDSSGPVPCIFFENFTYDQS |
| | AFDSGSHGLIPGPLKDTAHQLGLLAPPNKFQNVLKNDYMKPLVESPTEETSLIYVSQL |
| | ASPMCGDKDTLATEPPVPVHGSEYKRQMVVPGSLASPSLKEDNSLTSTVLLGQGEQ” |

11. SEQ ID NO: 11. PREDICTED: Equus caballus oncostatin M receptor (OSMR), transcript
variant X5, mRNA; NCBI Reference Sequence: XM_005604301.3

| | |
|---|---|
| LOCUS | XM_005604301 4622 bp mRNA linear MAM 23-JAN-2018 |
| DEFINITION | PREDICTED: Equus caballus oncostatin M receptor (OSMR), transcript |
| | variant X5, mRNA. |
| ACCESSION | XM_005604301 |
| VERSION | XM_005604301.3 |
| DBLINK | BioProject: PRJNA19129 |
| KEYWORDS | RefSeq. |
| SOURCE | Equus caballus (horse) |
| ORGANISM | Equus caballus |
| FEATURES | Location/Qualifiers |
| source | 1..4622 |
| | /organism=“Equus caballus” |
| | /mol_type=“mRNA” |
| | /isolate=“Twilight” |
| | /db_xref=“taxon:9796” |
| | /chromosome=“21” |
| | /sex=“female” |
| | /breed=“thoroughbred” |
| gene | 1..4622 |
| | /gene=“OSMR” |
| | /note=“Derived by automated computational analysis using |
| | gene prediction method: Gnomon. Supporting evidence |
| | includes similarity to: 1 Protein, and 100% coverage of |
| | the annotated genomic feature by RNAseq alignments, |
| | including 41 samples with support for all annotated |
| | introns” |
| | /db_xref=“GeneID:100066819” |
| CDS | 238..2967 |
| | /gene=“OSMR” |
| | /codon_start=1 |
| | /product=“oncostatin-M-specific receptor subunit beta |
| | isoform X4” |
| | /protein_id=“XP_005604358.1” |
| | /db_xref=“GeneID:100066819” |
| | /translation=“MVFQIEISRINTSNVIWVDYYSTTVKWNQVLHWSWESELPLECA |
| | THFVRIRSRVDDARVPEPRFWSNWSSWKEVNEQNSLGHKASSVFPVDKLVEEGSNVTI |
| | CYISRSHQNNVSCYLDGVRMHVEQLDPNVSAFHVNNVAFVRETGTNIFCEMDPKDDAG |
| | TVLFVSKVLEEPKDFSCETQDFRTLHCTWDPGSDTSLVRKQPYQSYTLFESFSGEKRL |
| | CEHKNWCDWQIAQDSQEMYNFTLVAENYLRKRSVNILFNLTHRVRPMTPKVFFKNISA |
| | TNATMTWRVHSTGNHRTLLCQIELHGEGKVIQQHNASIKVNGEYLLSELEPDTQYMAR |
| | VHCGDANHFWKWSEWSGENFTTLEAAPSEAPDVWRDVKSELGNRTVTLFWKPLSKSHA |
| | NGKIRFYNVIVENLDKPSELEPVPIPAPANHTKLALDQCSYRIHVTANNSVGTSPASV |
| | IVISGDPGNQVEEERVNVTADGESLSWKPQSGDVTGYVVEWCDRPQDGSCDLQWENLG |
| | PNTTSTVISSDAFRPGVRYNFRIYGISTKRIAYLLEQKTGYSQELAPLVNPQVVMSNL |
| | TSHSFTLSWKDYPTETQRSFIRGYHVYLKPKEGQCHPGFEKAALSDNSVCCKYKIDKP |
| | EQKTFVVENLQPESIYEFFVTPFTSVGEGPLDAFTKVTTPDEHSHLLIRIILPTVFCV |
| | VLIMIYCYLNSQWMKEKCYPDIPDPYKSSVLSLIKSKENPHLTIMSVKDCIPDAIEVI |
| | NKPEGSKIQFSGTRNSLTETEITKPAYLYLLPAEKNYSGPGPCICFENFTYNQAASNS |
| | GSCGHVPVPPTAPSQRGLLTSLENLLKALEKNYMNSQGEIPAGETTLNYVSQLASPMS |
| | GDKDSLPTDPPVPALCSEYKMQMAIPLGLASPPLSENSSVSSVTLLDQGDHHH” |
| ---- | |

12. SEQ ID NO: 12. PREDICTED: Felis catus oncostatin M receptor (OSMR), transcript
variant X1, mRNA; NCBI Reference Sequence: XM_023238963.2

| | |
|---|---|
| LOCUS | XM_023238963 4678 bp mRNA linear MAM 09-NOV-2021 |
| DEFINITION | PREDICTED: Felis catus oncostatin M receptor (OSMR), transcript |
| | variant X1, mRNA. |
| ACCESSION | XM_023238963 |
| VERSION | XM_023238963.2 |
| DBLINK | BioProject: PRJNA773801 |
| KEYWORDS | RefSeq. |
| SOURCE | Felis catus (domestic cat) |
| ORGANISM | Felis catus |
| FEATURES | Location/Qualifiers |
| source | 1..4678 |
| | /organism=“Felis catus” |
| | /mol_type=“mRNA” |
| | /isolate=“Fca126” |
| | /db_xref=“taxon:9685” |
| | /chromosome=“A1” |
| | /sex=“female” |
| | /cell_type=“Fibroblast” |
| | /tissue_type=“Fibroblasts” |
| | /dev_stage=“adult” |
| gene | 1..4678 |
| | /gene=“OSMR” |
| | /note=“Derived by automated computational analysis using |
| | gene prediction method: Gnomon. Supporting evidence |
| | includes similarity to: 3 Proteins, and 100% coverage of |
| | the annotated genomic feature by RNAseq alignments, |
| | including 100 samples with support for all annotated |
| | introng” |
| | /db_xref=“GeneID:101096447” |
| | /db_xref=“VGNC:68659” |
| CDS | 142..3027 |
| | /gene=“OSMR” |
| | /codon_start=1 |
| | /product=“oncostatin-M-specific receptor subunit beta |
| | isoform X1” |
| | /protein id=“XP_023094731.2” |
| | /db_xref=“GeneID:101096447” |
| | /db_xref=“VGNC:68659” |
| | /translation=“MALFSAFQTTFLLALLSLKTYQSEVLSEPLSLAPESLEVSIDSA |
| | RQCLHLKWSVHNLAYHQELKMVFQIEISRIKTSNVIWVENYSTTVKRNQVLRWSWESK |
| | LPLECAKHSVRMRGAVDDAQVPELRFWSNWTSWEEVDVQSSLGHDPLFVFPKDKLVEE |
| | GSNVTICYVSRSHQNNISCYLEGVRMHGEQLDPNVCVFHLKNVPFIRETGTNIYCKAD |
| | QGDVIKGIVLFVSKVFEEPKDFSCETRDLKTLNCTWAPGSDAGLLTQLSQSYTLFESF |
| | SGKKTLCKYKSWCNWQVSPDSQEMYNFTLTAENYLRKRSVHLLFNLTHRVHPMAPFNV |
| | FVKNVSATNATMTWKVHSIGNYSTLLCQIELYGEGKVIQKQNVSVKVNGKHLMKKLEP |
| | STEYAAQVRCANANHFWKWSEWTRRNFTTAEAAPSEAPDVWRNVKSVQGHCVVTLFWK |
| | PLSKLQANGKILFYNVVLENLDRPSTLKLLSIPAPANGTELTLDRCSYQIHVTANNSV |
| | GTSPASVIAVSGDPGNKEVEEERVKGTEDGFCLSWKPQPGDVTGYVVEWCERPGDPLC |
| | DLQWKNLGPNTTSTVVSSDAFRPGVRYNFRIYGISTERIPYLLEKKTGYSQELVPSDN |
| | PQVLMSNITSYSFTLSWKDYAAESQPGFVQGYSLYLKSKAARCLPGSEKAVLSDDSIC |
| | CKYKIDNPKQKTFVVENLQPASFYEFFLTPYTSVGEGPQGAFTKVTTPDEYSHILIRI |
| | ILPMIFSILLIMILCYLKSQWMKEKCYPDIPDPYKSSVLSLIKSKENPRLTIMNVKDC |
| | IPDAIEVINKQEGTRKSLTETEPTKPTYLYLLPTEKSASGPGPCICFENFTYNQAASD |
| | SVSCGHVPVTPKAQPSQLGLLISSENVPKALGKNYVNSLGEIPAGETNLNYVSQLASP |
| | MCGDKSSLPINPPEPALCSEYKTQMAIPMGLASPPSITLLDEGEHDH” |
| polyA_site | 4678 |
| | /gene=“OSMR” |
| | /experiment=“COORDINATES: polyA evidence [ECO:0006239]” |
| --- | |

1. Self-amplifying RNA (saRNA) that encodes a fusion protein comprising canine IL-31 fused to Lumazine synthase or canine IL-31 fused to diphtheria toxoid; or that encodes a protein comprising IL-31, an IL-31 variant, or an immunogenic fragment or epitope thereof; or that encodes a protein comprising IL31RA or that encodes a protein encoding Oncostatin M receptor β (OSMRβ) or an immunogenic fragment or epitope thereof; which comprises in any order:
(a) a first nucleic acid sequence comprising nsP1, nsP2, nsP3 and nsP4 that encode non-structural proteins of an alphavirus, and
(b) a second nucleic acid sequence that encodes IL-31, the IL-31 variant, or the immunogenic fragment or epitope thereof, and/or a second nucleic acid sequence that encodes IL3IRA and/or Oncostatin M receptor β (OSMRβ) or an immunogenic fragment or epitope thereof; and
(c) wherein the saRNA comprises a 5′ cap, a 5′ UTR upstream of the first nucleic acid sequence, a 26S promoter upstream of the second nucleic acid sequence, a 3′UTR, and a poly A tail;
which saRNA when incorporated into a cell expresses a protein comprising IL-31, the IL-31 variant, IL31RA, and/or Oncostatin M receptor β or an immunogenic fragment or epitope of any thereof.

2. The saRNA of claim 1, wherein the alphavirus is:
an Old World alphavirus selected from the group consisting of Sindbis virus (SINV), Chickungunya virus (CHIKV), Semliki Forest Virus (SFV), Ross River Virus (RRV), Sagiyama virus (SAGV), Getah virus (GETV), Middleburg virus (MIDV), Bebaru virus (BEBV), O'nyong nyong virus (ONNV), Ndumu (NDUV), and Barmah Forest virus (BFV); or
a New World alphavirus selected from the group consisting of Venezuelan Equine Encephalitis Virus (VEEV), western equine encephalitis virus (WEEV), and eastern equine encephalitis virus (EEEV).

3. The saRNA of claim 1 that comprises in the following order the 5′ cap, the 5′ UTR, nsP1, nsP2, nsP3, nsP4, the 26S promoter, the second nucleic acid sequence, the 3′UTR and the poly A tail.

4. The saRNA of claim 1, wherein the second nucleic acid sequence encodes wild-type canine, murine, equine or feline IL-31 as described by the accession numbers disclosed herein; and/or encodes wild-type canine, murine, equine or feline IL3IRA and/or Oncostatin M receptor β.

5. The saRNA of claim 1, wherein the second nucleic acid sequence encodes an IL-31 variant containing 1, 2, 3, 4, 5, 6, 7, 8, 9 or 10 insertions, substitutions, or deletions to the wild-type sequence of canine IL-31 (C7G0W1); murine IL-31 (Q6EAL8); equine IL-31 (F7AHG9), or feline IL-31 (A0A2I2UKP7·A0A2I2UKP7_FELCA) or wherein said variant has at least 50, 60, 70, 80, 90, 95, 99, 99.5, 99.9, or <100% sequence identity to the wild-type sequence of canine IL-31 (C7G0W1); murine IL-31 (Q6EAL8), equine IL-31 (F7AHG9), or feline IL-31 (A0A2I2UKP7·A0A2I2UKP7_FELCA); or wherein said variant is a homolog or isoform of the IL-31 as described by the accession numbers disclosed herein.

6. (canceled)

7. The saRNA of claim 1, wherein the second nucleic acid sequence encodes a variant of canine IL-31 comprising K133A, a variant of murine IL-31 comprising K138A, or a variant of equine IL-31 comprising K137A; or wherein the K at an aforementioned residue K133, K138 or K137 is replaced by an amino acid other than K.

8. The saRNA of claim 1, wherein the second nucleic acid sequence encodes a variant of canine IL-31 having a single substitutional mutation K133A; a variant of murine IL-31 having a single substitutional mutation K138A; a variant of equine IL-31 having a single substitutional mutation K137A; or a variant of a sequence described by the accession numbers disclosed herein, respectively, having a single substitutional mutation at K133, K138 or K137 which replaces said residue with an amino acid other than K.

9. (canceled)

10. The saRNA of claim 1, wherein the second nucleic acid sequence encodes IL-31, an IL-31 variant, or a fragment or epitope thereof that does not bind to the cytokine binding domain of IL31RA and/or to oncostatin M receptor (OSMR).

11.-16. (canceled)

17. The saRNA of claim 1, which further comprises at least one viral capsid enhancer or downstream loop (“DLP”) which optionally is immediately downstream of the 5′UTR or of the 26S promoter.

18. (canceled)

19. The saRNA of claim 1 which comprises at least one internal ribosome entry site (IRES) that is upstream of the second nucleic acid sequence encoding IL-31, its variants, fragments or epitopes.

20.-21. (canceled)

22. A composition comprising the saRNA of claim 1 in combination with a pharmaceutically acceptable carrier or excipient.

23. (canceled)

24. The composition of claim 22, wherein the pharmaceutically acceptable carrier or excipient comprises lipid inorganic nanoparticles (LION).

25. The composition of claim 22 in a form suitable for oral or mucosal administration.

26. The composition of claim 22 in a form suitable for intravenous, intramuscular or other parenteral administration.

27.-28. (canceled)

29. The composition of claim 22 in the form of a nanoemulsion comprising a hydrophobic core that comprises a mixture of a liquid oil and one or more inorganic nanoparticles, one or more lipids and, optionally, one or more surfactants.

30. A method for reducing the severity of atopic dermatitis in a mammal comprising: administering to a subject in need thereof an saRNA (self-amplifying RNA) that encodes IL-31, an IL-31 variant thereof, IL31RA, and/or Oncostatin receptor β or an immunogenic fragment, epitope, or variant of any thereof, according to claim 1.

31. The method of claim 30, wherein the mammal is a canine and the IL-31, IL-31 variant or immunogenic fragments thereof are derived from canine IL-31, canine IL3IRA, and/or canine Oncostatin M receptor β.

32. (canceled)

33. The method of claim 30, wherein the atopic dermatitis in an early phase characterized by pruritus and by abnormal scratching and/or licking, and/or by infiltration of TH2 lymphocytes upon examination of biopsied skin, but with normal looking skin with substantially no redness.

34. The method of claim 30, wherein the atopic dermatitis is in an acute phase characterized by abnormal skin redness and skin excoriation due to scratching and/or by comorbid infections.

35. The method of claim 30, wherein the atopic dermatitis is in a chronic stage characterized by thickening of the skin, lichenification, microbial infection, and/or darkening of affected skin.

36.-50. (canceled)


SEQUENCE LISTING (12 sequences)

SEQ ID NO: 1 - protein, 159 aa, Canis lupus
  source 1..159: note = RefSeq: NP_001159386.1; NM_001165914.1; note = subspecies familiaris
MLSHTGPSRFALFLLCSMETLLSSHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 2 - protein, 163 aa, Mus musculus
  source 1..163: note = EMBL; AY509149; AAS82846.1
MIFHTGTTKPTLVLLCCIGTWLATCSLSFGAPISKEDLRTTIDLLKQESQDLYNNYSIKQASGMSADESIQLPCFSLDREALTNISVIIAHLEKVKVLSENTVDTSWVIRWLTNISCFNPLNLNISVPGNTDESYDCKVFVLTVLKQFSNCMAELQAKDNTTC

SEQ ID NO: 3 - protein, 179 aa, Equus caballus
  source 1..179: note = XP_023503836.1
ATARTRAPGRGIGSRRLWPQRRRRSRKPRLPGSTRFALFLLCCLGTLMFSHTGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLEHRAQ

SEQ ID NO: 4 - protein, 213 aa, Felis catus
  source 1..213: note = XM_045042055.1; XP_044897990.1
MFKAKQNGDEAFDSPYSLWLSGPSPHTGMGILQKFPKWLQLGWAGGVWPRGPQHHFLSPGPARFALFLLCCMETLLPSHMAPTHRLQPSDVRKIILELRPMSKGLLQDYLKKEIGLPESNHSSLPCLSSDSQLPHINGSAILPYFRAIRPLSDKNTIDKIIEQLDKLKFQREPEAEVSMPADNFERKNFILAVLQQFSACLEHVLQSLNSGPQ

SEQ ID NO: 5 - protein, 361 aa, Canis lupus
  source 1..361: note = XM_038658911.1; XP_038514839.1; note = subspecies familiaris
HTTPSWWWNCBINLMNIHGOVNUCCOREXMTEXTMMWAKVLWMLLLLCKLSLAVLPAKPENISCIFYYEENFTCTWSPAEKEASYTWYKVKRTYSYGYKSDICSTDNSTRGNHASCSFLPPTITNPDNYTIQVEAQNAADGIMKSDITYWNLDAIMKIEPPEIFSVKSVLGIKRMLQIKWIRPVLAPHSSTLKYTLATWRGDDFKDKLNLKESDDPVNMEEDQVLKPYSAPTDFIDKLVVNFENFLEEVSTEELAGKSQENILKEEKNKHVTSPYCLHHPPISTEIPQRKPQQLCSRIPEGTCSETKEQLFSSAVQSLGPDHLCEEGEPNPYLKNSVTTREFVCLKNFKTKPKEKFSVAVA

SEQ ID NO: 6 - protein, 716 aa, Mus musculus
  source 1..716: note = NM_139299.2; NP_647460.2
MWTLALWAFSFLCKFSLAVLPTKPENISCVFYFDRNLTCTWRPEKETNDTSYIVTLTYSYGKSNYSDNATEASYSFPRSCAMPPDICSVEVQAQNGDGKVKSDITYWHLISIAKTEPPIILSVNPICNRMFQIQWKPREKTRGFPLVCMLRFRTVNSSHWTEVNFENCKQVCNLTGLQAFTEYVLALRFRFNDSRYWSKWSKEETRVTMEEVPHVLDLWRILEPADMNGDRKVRLLWKKARGAPVLEKTFGYHIQYFAENSTNLTEINNITTQQYELLLMSQAHSVSVTSFNSLGKSQEAILRIPDVHEKTFQYIKSMKAYIAEPLLVVNWQSSIPAVDTWIVEWLPEAAMSKFPALSWESVSQVTNWTIEQDKLKPFTCYNISVYPVLGHRVGEPYSIQAYAKEGTPLKGPETRVENIGLRTATITWKEIPKSARNGFINNYTVFYQAEGGKELSKTVNSHALQCDLESLTRRTSYTVWVMASTRAGGTNGVRINFKTLSISVFEIVLLTSLVGGGLLLLSIKTVTFGLRKPNRLTPLCCPDVPNPAESSLATWLGDGFKKSNMKETGNSGDTEDVVLKPCPVPADLIDKLVVNFENFLEVVLTEEAGKGQASILGGEANEYVTSPSRPDGPPGKSFKEPSVLTEVASEDSHSTCSRMADEAYSELARQPSSSCQSPGLSPPREDQAQNPYLKNSVTTREFLVHENIPEHSKGEV

SEQ ID NO: 7 - protein, 745 aa, Equus caballus
  source 1..745: note = XM_023625297.1
MAIQLSHQPAXDSXKTMWAWTLWMLPLLCKFSLAVLLAKPENISCIFYYERNFTCTWSPEKEASDTWYTVKRTYCFGRKSDHCTTGNSTSTARTSCSFYPPPITIPDNYTIQVEAQNADGIIKSDITQWRLDTIVKTEPPEIYSVKPVLGIKRMVQIKWKRPVLAPKSTTLKYRLRFRTVNSAGWMEVNFKEGSNGDTTYNLTGLQAFTEYVVALQCVAEESVFWSGWSQEKMGTTEEEAPHGLDLWRVLRPAMVDGRRPVQLLWKKARGAPVLEKTLGYNIQYFPENNTNLTKTVNTTNQQLDLYLGGETYWVSVISYNSLGESSLATLRIPAIDEKPFQCIEAMHSCLTQDQLVVEWQSSASEVDTWMVEWFPVLDSEPSIFSWESVSGARNWTIQQDELKPFWCYNISVYPMLQDQVGEPYSIQAYVKEGIPSVGPVSKVENIGVKTVTITWKEIPTSRRNGFISNYTIFYQAEDGKEFSKTVNSSILQYGLESLTRMTSYTVQVMASTSAGGINGTRINFKTLSISVFEIFLITSLVGGSLLILIIPTVAYGLKKPNKLKHLCWPDVPNPAESSIAMWRGDNFEDKLNVKEFDDSVNMEEERILKPCSVPSDLIDKLVVDFENFLEEFSTEEPGKGQENILGGEKNEYVTSPYRPYCPTGKHFKELPLATEIPSRKSQYLSSGVPEGTSSEDKEQLLSSASSLGLDHLCEEGAPNPYLKNSVTTREFLVPEKLPDQTKREV

SEQ ID NO: 8 - protein, 723 aa, Felis catus
  source 1..723: note = XM_019834303.3; XP_019689862.3
MMWAHALWTLLLLCKFSLAVLPAKPENISCVFYYEENFTCTWSPEKEASYTWYKVKRTYSYGYKSDICPSDNSTRGNHTFCSFLPPTITNPDNYTIQVEAQNADGIIKSDITHWSLDAITKIEPPEIFSVKPVLGVKRMVQIKWIRPVLAPVSSTLKYTLRFKTVNSAYWMEVNFTKEDIDRDETYNLTGLQAFTEYVLALRCATKESMFWSGWSQEKMGTTEEEAPHGLDLWRVLRPATVDGRRLVQLMWKKASGAPVLEKALGYNIWYFPENSTNLTKTLNTTNEKLELYLGGKTYWVCVVSYNSLGESPVATLRIPAIDEKSFQCIEAMEACLTQDQLVVEWRSSAPEVDTWMVEWFPDLDSEPSTFSWESVSQATNWTIKQDELKPFWCYNISVYPVLQDRVGKPFSIQAYVREGIPSAGPVTQVDNIGVKTVTITWKEIPKSQRNGFITNYTIFYQAEDGKEFSKTVNSNILQYDLESLTRKTSYSLQVMASTSAGGINGTTMNFKTLSISILEIFLIISLVGGGLLILIILSVAYGLKKPNRLKHLCWPDVPNPAESSIATWRGDDFKDKINLKESDDPVNMEEDRVLKPYSSPRDLIDKLVVNFENFLEDVSTEELGKGQENILREEKNEYVTSPYRPYCPPISTEIPQRKSQQLCSRIPEGICLETTEQLLSSVPNLGPDRICEEGEPNPYLKNSVTTREFLTSEKLPEQTKREV

SEQ ID NO: 9 - protein, 783 aa, Canis lupus
  source 1..783: note = XM_038663878.1; XP_038519806.1; note = subspecies familiaris
MQGQQLDPNVSMFNLHNVAFIRETGTNIYCKVDRGDDIKGIVLFVSKILEEPKDFSCETRDFQTLSCTWDPGRDTGLLKQLPQSYTLFESFSGKKTLCKHKGWCNWQVAPESQEMYNFTLTAENYLRKRSVHILFNLTHRVHPMAPFNVLFKDVSVTNATMTWKVHSTGNYYTLLCQVELYGEGKVIQKHNVSVKVNGELVLSGLEPDTEYSAQVRCANANHFWKWSEWTRQNFTTVEAAPSEAPDVWRNVKSVQGYCVVTLFWKPLSRLQANGEILFYNIVVEDLDRPSGLQLLSVPAPANRTELTLDQRAYQIHVTANNSVGTSPASVLVVSGDPGDEEVEEERVKGTEDGFSLSWKPQPGNITGYIVEWCDRPRDPLCDLQWKHLGPNTTSTVISSDAFRPGVRYNFRIYGISTEMVPYLLEKKTGYSQELAPSDNPRVVMSNLTSHSFTLSWKDYSTESQPSFIRGYHVYLKSKAGQCHPGSEKAVLSDDSVCCKYKIDDPKQKMFVVGNLQPESFYEFLVTPYSAVGEGPHGAFTKVTTPDEYSPMLIRIILPMIFCILLIMILCYLKSQWMKEKCYPDIPDPYKSSVLSLIKSKENPRLTIMNIKDCVPDTIEVVNKHEGTGKSPTEPEAAKPTYLYLLPAEESYSGPGPCICFENFTYNQAASDSASCGHVPVPTKAPSQLGLLTSSENLLTALEKNCMNSLEEIPAGESSLNYVSQVASPVSGDKSSLPTNPPQPELCSEYKMQMAIPLGLAAPSSSGSSSLSSITLLDEGEHYH

SEQ ID NO: 10 - protein, 970 aa, Mus musculus
  source 1..970: note = AB015978.1; BAA33725.1
MAFSVVLHPAFLLAVLSLRASRSEVLEEPLPLTPEIHKVSFQLKLQEVNLEWTVPALTHEELNMIFQIEISRLNISNTIWVENYSTTVKREEAVRWNWTSDIPLECVKHFIRIRALVDDTKSLPQSSWGNWSSWKEVNAKVSVEPDKSLIFPKDKVLEEGSNVTICLMYGQNVYNVSCKLQDEPIHGEQLDSHVSLLKLNNVVFLSDTGTNINCQATKGPKRIFGTVLFVSKVLEEPKNVSCETRDFKTLDCSWEPGVDTTLTWRKQRFQNYTLCESFSKRCEVSNYRNSYTWQITEGSQEMYNFTLTAENQLRKRSVNINFNLTHRVHPKAPQDVTLKIIGATKANMTWKVHSHGNNYTLLCQVKLQYGEVIHEHNVSVHMSANYLFSDLDPDTKYKAFVRCASANHFWKWSDWTQKEFSTPETAPSQALDVWRQVWSENGRRIVTLFWKPLLKSQANGKIISYNIVVENEAKPTESEHYCVWAPALSTNLSLDLQPYKIRITANNSMGASPESLMVLSNDSGHEVKEKTIKGIKDAFNISWEPVSGDTMGYVVDWCAHSQDQRCDLQWKNLGPNTTSTTITSDDFKPGVRYNFRIFERSVEHKARLVEKQRGYTQELAPLVNPKVEIPYSTPNSFVLRWPDYDSDFQAGFIKGYLVYVKSKEMQCNQPWERTLLPDNSVLCKYDINGSETKTLTVENLQPESLYEFFVTPYTSAGPGPNETFTKVTTPDARSHMLLQIILPMTLCVLLSIIVCYWKSQWVKEKCYPDIPNPYKSSILSLIKSKKNPHLIMNVKDCIPDVLEVINKAEGSKTQCVGSGKLHIEDVPTKPPIVPTEKDSSGPVPCIFFENFTYDQSAFDSGSHGLIPGPLKDTAHQLGLLAPPNKFQNVLKNDYMKPLVESPTEETSLIYVSQLASPMCGDKDTLATEPPVPVHGSEYKRQMVVPGSLASPSLKEDNSLTSTVLLGQGEQ

SEQ ID NO: 11 - protein, 909 aa, Equus caballus
  source 1..909: note = XM_005604301.3; XP_005604358.1
MVFQIEISRINTSNVIWVDYYSTTVKWNQVLHWSWESELPLECATHFVRIRSRVDDARVPEPRFWSNWSSWKEVNEQNSLGHKASSVFPVDKLVEEGSNVTICYISRSHQNNVSCYLDGVRMHVEQLDPNVSAFHVNNVAFVRETGTNIFCEMDPKDDAGTVLFVSKVLEEPKDFSCETQDFKTLHCTWDPGSDTSLVRKQPYQSYTLFESFSGEKRLCEHKNWCDWQIAQDSQEMYNFTLVAENYLRKRSVNILFNLTHRVRPMTPKVFFKNISATNATMTWRVHSTGNHRTLLCQIELHGEGKVIQQHNASIKVNGEYLLSELEPDTQYMARVHCGDANHFWKWSEWSGENFTTLEAAPSEAPDVWRDVKSELGNRTVTLFWKPLSKSHANGKIRFYNVIVENLDKPSELEPVPIPAPANHTKLALDQCSYRIHVTANNSVGTSPASVIVISGDPGNQVEEERVNVTADGFSLSWKPQSGDVTGYVVEWCDRPQDGSCDLQWENLGPNTTSTVISSDAFRPGVRYNFRIYGISTKRIAYLLEQKTGYSQELAPLVNPQVVMSNLTSHSFTLSWKDYPTETQRSFIRGYHVYLKPKEGQCHPGFEKAALSDNSVCCKYKIDKPEQKTFVVENLQPESIYEFFVTPFTSVGEGPLDAFTKVTTPDEHSHLLIRIILPTVFCVVLIMIVCYLNSQWMKEKCYPDIPDPYKSSVLSLIKSKENPHLTIMSVKDCIPDAIEVINKPEGSKIQFSGTRNSLTETEITKPAYLYLLPAEKNYSGPGPCICFENFTYNQAASNSGSCGHVPVPPTAPSQRGLLTSLENLLKALEKNYMNSQGEIPAGETTLNYVSQLASPMSGDKDSLPTDPPVPALCSEYKMQMAIPLGLASPPLSENSSVSSVTLLDQGDHHH

SEQ ID NO: 12 - protein, 961 aa, Felis catus
  source 1..961: note = XM_023238963.2; XP_023094731.2
MALFSAFQTTFLLALLSLKTYQSEVLSEPLSLAPESLEVSIDSARQCLHLKWSVHNLAYHQELKMVFQIEISRIKTSNVIWVENYSTTVKRNQVLRWSWESKLPLECAKHSVRMRGAVDDAQVPELRFWSNWTSWEEVDVQSSLGHDPLFVFPKDKLVEEGSNVTICYVSRSHQNNISCYLEGVRMHGEQLDPNVCVFHLKNVPFIRETGTNIYCKADQGDVIKGIVLFVSKVFEEPKDFSCETRDLKTLNCTWAPGSDAGLLTQLSQSYTLFESFSGKKTLCKYKSWCNWQVSPDSQEMYNFTLTAENYLRKRSVHLLFNLTHRVHPMAPFNVFVKNVSATNATMTWKVHSIGNYSTLLCQIELYGEGKVIQKQNVSVKVNGKHLMKKLEPSTEYAAQVRCANANHFWKWSEWTRRNFTTAEAAPSEAPDVWRNVKSVQGHCVVTLFWKPLSKLQANGKILFYNVVLENLDRPSTLKLLSIPAPANGTELTLDRCSYQIHVTANNSVGTSPASVIAVSGDPGNKEVEEERVKGTEDGFCLSWKPQPGDVTGYVVEWCERPGDPLCDLQWKNLGPNTTSTVVSSDAFRPGVRYNFRIYGISTERIPYLLEKKTGYSQELVPSDNPQVLMSNLTSYSFTLSWKDYAAESQPGFVQGYSLYLKSKAARCLPGSEKAVLSDDSICCKYKIDNPKQKTFVVENLQPASFYEFFLTPYTSVGEGPQGAFTKVTTPDEYSHILIRIILPMIFSILLIMILCYLKSQWMKEKCYPDIPDPYKSSVLSLIKSKENPRLTIMNVKDCIPDAIEVINKQEGTRKSLTETEPTKPTYLYLLPTEKSHSGPGPCICFENFTYNQAASDSVSCGHVPVTPKAQPSQLGLLTSSENVPKALGKNYVNSLGEIPAGETNLNYVSQLASPMCGDKSSLPTNPPEPALCSEYKTQMAIPMGLASPPSITLLDEGEHDH